(19) United States
(12) Patent Application Publication
(10) Pub. No.: US 2004/0180852 A1
(43) Pub. Date: Sep. 16, 2004

(54) Use of multivalent glycodendrimers to inhibit the activity of human immunodeficiency virus

(76) Inventors: Cara-Lynne Schengrund, Hummelstown, PA (US); Richard D. Kensinger, Cresco, PA (US); Andrew Rosa Borges, Hershey, PA (US)

Correspondence Address: GIFFORD, KRASS, GROH, SPRINKLE, ANDERSON & CITKOWSKI, PC, 280 N OLD WOODARD AVE, SUITE 400, BIRMINGHAM, MI, 48009, US

(21) Appl. No.: 10/796,707

(22) Filed: Mar. 9, 2004

Related U.S. Application Data
(60) Provisional application No. 60/454,210, filed on Mar. 10, 2003.
(60) Provisional application No. 60/545,072, filed on Feb. 17, 2004.

(51) Int. Cl. C07H 5/04; A61K 31/715

(52) U.S. Cl. 514/54; 536/18.7

(57) ABSTRACT

The present invention relates to compositions and methods for inhibiting the interaction of a ligand and receptor. In particular, compositions are methods are provided for inhibition of interaction between a virus, especially HIV, and a receptor. Inhibition of such ligand/receptor interaction is advantageous in inhibiting infection of a cell with a virus. Further, an inhibitor of ligand/receptor interaction serves as a standard in assays of inhibition in vitro or in vivo. In a particular embodiment, a composition according to the invention includes a compound having the formula: [(X)—(Y)]ₚ-Z where X is

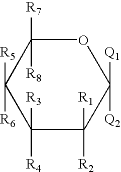

and where Q1 and Q2 are each independently H, a bond to Y, or a bond to Z, where at least one of Q1 or Q2 is a bond to Y or a bond to Z; where R1, R4, R6 and R8 are each H; R2, R3, and R5 are each independently OH, OSO3D, or OPO3D, and R7 is CH20H, CH2OSO3D or CH2OPO3D; where D is H or a cation selected from the group consisting of: alkali metal cations, alkaline earth metal cations, ammonium cations, quaternary ammonium cations and amine cations; and where at least one of R2, R3, R5 and R7 is a sulfur or phosphate containing group; where Y is an optional linker, Z is a multivalent support, and p is an integer in the range of 1-2000 inclusive.

### RELATED APPLICATIONS

This application claims priority of U.S. Provisional Patent Applications 60/454,210 filed Mar. 10, 2003, and 60/545,072, filed Feb. 17, 2004, both of which are incorporated herein by reference.

### GOVERNMENT SPONSORSHIP

This work was supported by the National Institute of Health under Grant Nos. NS40231 and NS11184. Accordingly, the U.S. government may have certain rights in this invention.

### FIELD OF THE INVENTION

The present invention generally relates to compositions and methods inhibiting ligand/receptor interaction. In a particular embodiment, the present invention relates to compounds and methods for inhibiting HIV.

### BACKGROUND OF THE INVENTION

The problem of weak affinity in protein-carbohydrate interactions may be addressed by displaying multiple copies of both the carbohydrate ligands and their receptors on the surfaces of interacting cells, such that the many weak interactions reinforce one another in a cooperative manner. This phenomenon has been termed the “multivalent” or “glycoside cluster effect.” In addition to enhancing binding strength, multivalency also amplifies binding selectivity.

Glycosphingolipids (GSLs) are good examples of clustering, in that the polar carbohydrate head groups are often packed together on the cell surface in lipid rafts or “detergent insoluble membranes” where, among other things, they can serve as binding sites for various bacteria, viruses, cells and bacterial toxins.

Although the primary protein receptors for human immunodeficiency virus (HIV) are believed to be the cell surface CD4 molecules, cell surface GSLs, galactosyl ceramide (GalCer) and its 3′-sulfated derivative, SGalCer, may act as alternate receptors for HIV-1 gpl20. For instance, antibodies to GalCer inhibited HIV-1 entry into glioma and human neuroblastoma cells, a GalCer binding site was mapped to the V3 loop of gpl20 (binding protein present on the surface of HIV-1 virions), rgpl20 adhered to sulfatide (gal-3-sulfatecer), and HIV was unable to fuse with GSL-depleted cells.

HIV is very complex and presents many different potential targets for therapeutic intervention. The key to controlling HIV is to block HIV-1 entry, and/or elicit an immune response that is able to kill the virus. The heavily glycosylated gpl20, highly variable loops, hidden co receptor binding site(s), buried fusion peptides, and unfaithful replication of reverse transcriptase have all contributed to the inability to produce an effective vaccine to date. Much research has been directed to the development of therapeutics for treatment and prevention of HIV infection. While there are several compounds in various stages of clinical trials which relate to HIV infection, there remains a great need for further development of compounds in this area.

### SUMMARY OF THE INVENTION

Provided by the present invention is composition including a compound having the formula:

[(X)—(Y)]ₚ-Z where X is

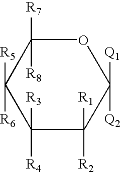

and where Q1 and Q2 are each independently H, a bond to Y, or a bond to Z, where at least one of Q1 or Q2 is a bond to Y or a bond to Z; where R1, R4, R6 and R8 are each H; R2, R3, and R5 are each independently OH, OSO3D, or OPO3D, and R7 is CH2OH, CH2OSO3D or CH2OPO3D; where D is H or a cation selected from the group consisting of: alkali metal cations, alkaline earth metal cations, ammonium cations, quaternary ammonium cations and amine cations; and where at least one of R2, R3, R5 and R7 is a sulfur or phosphate containing group; where Y is an optional linker, Z is a multivalent support, and p is an integer in the range of 1-2000 inclusive.

Also provided is a composition including a compound having the formula:

[(X)—(Y)]ₚ-Z where X is

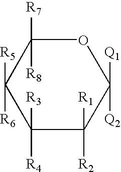

and where Q1 and Q2 are each independently H, a bond to Y, or a bond to Z, where at least one of Q1 or Q2 is a bond to Y or a bond to Z; where R1, R4, R5 and R8 are each H; R2, R3, and R6 are each independently OH, OSO3D, or OPO3D, and R7 is CH2OH, CH2OSO3D, CH2OPO3D; where D is H or a cation selected from the group consisting of: alkali metal cations, alkaline earth metal cations, ammonium cations, quaternary ammonium cations and amine cations; and where at least one of R2, R3, R6 and R7 is a sulfur or phosphate containing group; where Y is an optional linker, Z is a multivalent support, and p is an integer in the range of 1-2000.

Further provided is a composition including a compound having the formula:

[(X)—(Y)]ₚ-Z where X is

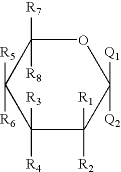

and where Q1 and Q2 are each independently H, a bond to Y, or a bond to Z, where at least one of Q1 or Q2 is a bond to Y or a bond to Z; where R1, R2, R3, R4, R5 and R6 are each independently H, OH, COOH, sialic acid, OSO3D, or OPO3D; R7 and R8 are each independently H, OH, COOH, sialic acid, CH2OH, CH2OSO3D, CH2OPO3D, OSO3D, or OPO3D, where D is H or a cation selected from the group consisting of: alkali metal cations, alkaline earth metal cations, ammonium cations, quaternary ammonium cations and amine cations; and where at least one of R1, R2, R3, R4, R5, R6, R7 and R8 is a sulfur or phosphate containing group; where Y is an optional linker, Z is a multivalent support, and p is an integer in the range of 1-2000. Optionally, p is 2-2000 inclusive or 3-525 inclusive and at least one of R1, R2, R3, R4, R5, R6, R7 and R8 on each X independently is a sulfur or phosphate containing group.

Provided is a composition including a compound having the formula:

[(X)—(Y)]ₚ-Z where X is

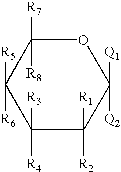

and where Q1 and Q2 are each independently H, a bond to Y, or a bond to Z, where at least one of Q1 or Q2 is a bond to Y or a bond to Z; where the bond to Y is a bond to an atom selected from the group consisting of: S, P, N, or C; where R1, R2, R3, and R4 are each independently H, OH, COOH, OSO3D, or OPO3D; where R7 and R8 are each independently H, CH2OH, COOH, CH2OSO3D, CH2OPO3D, OSO3D, or OPO3D; where R5 and R6 are each independently H or a saccharyl group having the formula:

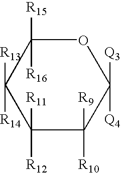

where one of the variables Q3 and Q4 is O and the other is H; where R9, R10, R11, R12, R13 and R14 are each independently H, OH, COOH, sialic acid, OSO3D, or OPO3D; where R15 and R16 are each independently H, OH, COOH, sialic acid, CH2OH, CH2OSO3D, CH2OPO3D, OSO3D, or OPO3D; where D is H or a cation selected from the group consisting of: alkali metal cations, alkaline earth metal cations, ammonium cations, quaternary ammonium cations and amine cations; where at least one of R1, R2, R3, R4, R5, R6, R7, R8 R9, R10, R11, R12, R13, R14, R15 and R16 is a sulfur or phosphate containing group; and where Y is an optional linker, Z is a multivalent support, and p is an integer in the range of 1-2000.

A composition is provided that includes a compound having the formula: [(X)—(Y)]ₚ-Z where X is

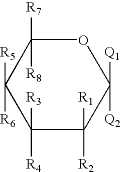

where Q1 and Q2 are each independently H or a bond to Y, where at least one of Q1 or Q2 is a bond to Y; and where R1, R2, R3, and R4 are each independently H, OH, COOH, sialic acid, OSO3D, or OPO3D; R7 and R8 are each independently H, COOH, sialic acid, CH2OH, CH2OSO3D, CH2OPO3D, OSO3D, or OPO3D; and R5 and R6 are each independently H or a saccharyl group having the formula:

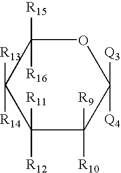

where one of the variables Q3 and Q4 is oxygen and the other is H; and where R9, R10, R11, R12, R13 and R14 are each independently H, OH, COOH, sialic acid, OSO3D, or OPO3D; and R15 and R16 are each independently H, OH, COOH, sialic acid, CH2OH, CH2OSO3D, CH2OPO3D, OSO3D, or OPO3D; where D is H or a cation selected from the group consisting of: alkali metal cations, alkaline earth metal cations, ammonium cations, quaternary ammonium cations and amine cations; and where at least one of R9, R10, R11, R12, R13, R14, R15 and R16 is a sulfur or phosphate containing group; and where Y is an optional linker, Z is a multivalent support, and p is an integer in the range of 1-2000.

Also described is an inventive composition including a compound having the formula: [(X)—(Y)]ₚ-Z where X is

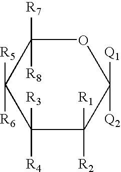

where Q1 and Q2 are each independently H or a bond to Y, where at least one of Q1 or Q2 is a bond to Y; and where R1, R2, R3, and R4 are each independently H, OH, COOH, sialic acid, OSO3D, or OPO3D; R7 and R8 are each independently H, OH, COOH, sialic acid, CHO2H, CH2OSO3D, CH2OPO3D, OSO3D, or OPO3D; and R5 and R6 are each independently H or a saccharyl group having the formula:

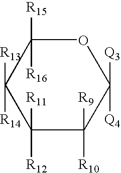

where one of the variables Q3 and Q4 is oxygen and the other is H; where R9, R10, R11, and R12 are each independently H, OH, COOH, sialic acid, OSO3D, or OPO3D; where R15 and R16 are each independently H, OH, COOH, sialic acid, CHO2H, CH2OSO3D, CH2OPO3D, OSO3D, or OPO3D; where R13 and R14 are each independently H or a saccharyl group having the formula:

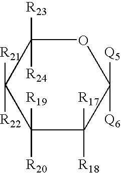

where one of the variables Q5 and Q6 is oxygen and the other is H; and where R17, R18, R19, R20, R21 and R22 are each independently a saccharyl group, H, OH, COOH, sialic acid, OSO3D, or OPO3D; and R23 and R24 are each independently a saccharyl group, H, OH, COOH, sialic acid, CH2OH, CH2OSO3D, CH2OPO3D, OSO3D, or OPO3D; where D is H or a cation selected from the group consisting of: alkali metal cations, alkaline earth metal cations, ammonium cations, quaternary ammonium cations and amine cations; and where at least one of R1, R2, R3, R4, R5, R6, R7, R8 R9, R10, R11, R12, R13, R14, R15, R16 R17, R18, R19, R20, R21, R22, R23 and R24 is a sulfur or phosphate containing group; and where Y is an optional linker, Z is a multivalent support, and p is an integer in the range of 1-2000.

In the above described embodiments p is optionally in the range of 2-2000 inclusive or 3-525 inclusive, and at least one of the variables R2, R3, R6 and R7 on each X independently is a sulfur or phosphate containing group. In further options, each inventive composition has an average of 1-4, or 2-3, sulfur or phosphate containing groups per moiety X. Also optionally, Z is a dendrimer, a protein, a lipid, a carbohydrate, a synthetic polymer, a natural polymer or a combination thereof.

Inventive pharmaceutical compositions are detailed herein which include a composition as described above along with a pharmaceutical carrier or excipient.

Also described is a process for inhibiting interaction of a ligand and a receptor. An inventive process includes the steps of providing a composition as detailed herein and introducing the composition into an environment containing the ligand or the receptor, such that interaction of the ligand and the receptor is inhibited by the composition. The ligand component of an inventive process for inhibiting ligand/receptor interaction is a virus, a component of a virus, a cell, or a component of a cell. Further the receptor is a cell or a component of a cell. In particular, the virus is a virus of the family Retroviridae, and optionally an HIV. In a further option, the environment is a human or non-human animal body and the receptor is endogenous to the body of an individual subject while the ligand is exogenous. In a particular embodiment, the ligand is a sperm cell and the receptor is an ovum.

An inventive process for therapeutic or prophylactic anti-viral treatment is described including the steps of administering an effective amount of a composition as described herein to an individual human or non-human animal in need of such therapeutic or prophylactic anti-viral treatment. Optionally, the individual human or non-human animal is at risk of retroviral infection and further optionally the individual human or non-human animal is infected with a retrovirus.

A commercial package is provided which includes an inventive composition as detailed herein along with instructions for use.

### BRIEF DESCRIPTION OF THE DRAWINGS

FIG. 1 is a drawing illustrating aspects of compounds of the present invention.

FIG. 2 is a graph illustrating inhibitory properties of an inventive compound.

FIG. 3A is a graph illustrating inhibitory properties of an inventive compound.

FIG. 3B is a graph illustrating inhibitory properties of an inventive compound.

FIG. 3C is a graph illustrating inhibitory properties of an inventive compound.

FIG. 3D is a graph illustrating inhibitory properties of an inventive compound.

### DETAILED DESCRIPTION OF THE INVENTION

A composition of the present invention includes a compound having the formula: [(X)—(Y)]ₚ-Z

wherein X is a saccharide residue having sulfur and/or phosphate groups,

Y is an optional linker, Z is a multivalent support, and p is an integer in the range of 1-2000.

A saccharide residue included in an inventive composition is selected from the group monosaccharide, disaccharide, trisaccharide, and a saccharide including 4-10 monosaccharide units. Particularly preferred is a saccharide residue including at least one hexose residue, such as allose, altrose, glucose, mannose, gulose, idose, galactose, talose and combinations thereof. In some embodiments, a saccharide residue is a pentose, tetrose or triose. A saccharide residue may contain a combination of hexose, pentose, tetrose and triose residues. Further, in some embodiments, a saccharide residue may contain a 7-, 8-, 9, 10-, 11-, 12- or more carbon saccharide, such as sialic acid.

Preferred saccharide residues include pyranose forms. A saccharide residue includes D- and L-aldopyranoses, D- and L-aldofuranoses, D- and L-ketopyranoses and D- and L-ketofuranoses. In some embodiments, a saccharide residue further includes modified saccharide residues such as deoxy derivatives, including dexoyamine, deoxythio-, and deoxyhalo-saccharides. A saccharide residue further includes acid derivatives of saccharide residues described above, such as glucuronic acid and galacturonic acid. In addition, a saccharide residue includes glycosyl residues such as N-acetylneuraminic acid (sialic acid).

While some compounds described herein are referred to in terms of specific stereoisomer or anomer forms, specifically contemplated as within the scope of inventive compounds, compositions and components thereof, particularly the saccharide components thereof, are D- and L-stereoisomers, combinations thereof, α and β anomers and combinations thereof.

Saccharides may include substituents replacing an alcoholic hydroxy group or the hydrogen atom of an alcoholic hydroxy group of a saccharide or saccharide derivative. Such substituents include COOH, sialic acid, NHAc, C1-8 acyl, anhydro, C1-8 alkyl, NH2, halogen, OSO3D, OPO3D, a bond to Y, or a bond to Z, CH2OH, CH2OSO3D, or CH2OPO3D. Further, a substituent may be a saccharyl group as described below.

Particularly preferred are saccharides including an O-substituent, that is, in which a substituent replaces the hydrogen atom of an alcoholic hydroxy group of a saccharide or saccharide derivative. Examples of such substituents include alkyl-, acyl-, and phosphorus containing-groups. Preferred substituents include the sulfur moieties (DO)S(O)₂— or (O⁻)S(O)₂—, bonded to oxygen, where D is H or a cation. The phrase “sulfur or phosphate containing group” as used herein includes SO3-, SO3H, SO3D, PO3-, PO3H, or PO3D, where D is a cation. A cation D includes cations based on the alkali and alkaline earth metals, such as sodium, lithium, potassium, calcium, magnesium, and the like, as well as non-toxic ammonium, quaternary ammonium and amine cations including, but not limited to ammonium, tetramethylammonium, tetraethylammonium, methylamine, dimethylamine, trimethylamine, triethylamine, ethylamine, and the like.

Where multiple monosaccharide residues are included in a saccharide, they are preferably linked by α-O-glycosidic or β-O-glycosidic linkage. Further, S-, N- and C-glycosidic linkages are optionally included. A preferred optional glcycosidic link is an S-linkage since this is more resistant to glycosidases than a corresponding O-glycosidic bond between monosaccharide units. Typical bonds include (1→2), (1→3)-, (1→4)-, (1→5)-, (1→6)-, (2→3)- and (2→6) glycosidic linkages. One of skill in the art will recognize that where a monosaccharide having more than 6 carbons is present, further typical bonds are present, such as (2→8) for instance.

As indicated above, Y is an optional linker. Y is any linker or bond linking the saccharide moiety, X, to the multivalent support, Z, by a covalent bond. Suitable linkers are known in the art and include carbohydrate-based linkers, peptide or protein-based linkers, alkyl chains or groups including C₁-C₁₆ thioalkyl, oxyalkyl and unsubstituted alkyl groups.

Optionally, a link between X and Y is resistant to glycosidases, such as those endogenous to a human. For example, a thioglycosidic linkage is more resistant to glycosidases than a corresponding O-glycoside. An exemplary linker is a thiopropionic acid derivative as described and shown herein.

Further optionally, Y may include a component of a glycosphingolipid. For example, Y optionally includes ceramide such that X—Y includes sulfated galactosyl ceramide.

Z is a multivalent support to which X is bound indirectly, that is, by an intervening spacer or linker, Y. In an alternative embodiment, X is directly bound to the multivalent support.

A multivalent support allows binding of multiple groups, X. In particular, a multivalent support is indirectly attached, e.g. through a linker Y, or directly attached, e.g. by a covalent bond, to a number of groups, X. The particular number of groups, X, attached to the multivalent support is represented by p. The number p is an integer in the range of 1-2000 inclusive. A preferred range for the value of p is 2-500 inclusive. A further preferred range for the value of p is 3-100 inclusive. In some embodiments, a preferred range for p is 3-70 inclusive. Where the multivalent support is a dendrimer, the value of p is a multiple of an initial number of derivatized functional groups. Thus, p increases with each “generation” of a dendrimer as a function of the initial number of derivatized functional groups. For example, in an exemplary embodiment where the multivalent support is a dendrimer, p is selected from 4, 8, 16, 32, 64, et seq.

The particular number, p, for a given multivalent support depends on the number of sites available for attachment of moieties, X— or X—Y—. For instance, a dendrimer having 64 terminals to which a linker conjugate, X—Y—, or group X, may be attached has a maximum of 64 attachment sites available. Typically, the number of available sites will be equivalent to the number of moieties, X— or X—Y— actually attached. Optionally, the number of moieties, X— or X—Y— actually attached to the multivalent support Z is less than the number of sites available or capable of such attachment. Thus, the number of moieties, X— or X—Y— actually attached to the multivalent support Z may be expressed as, p=pt−pe, where pt is the total number of attachment sites available and pe is the number of attachment sites having no moiety X— or X—Y— actually attached.

A multivalent support is characterized by binding sites for X—Y— or X— groups, and may be of a variety of compositions. For example, a multivalent support is optionally a protein, a lipid, a carbohydrate, a synthetic polymer, a natural polymer or a combination thereof.

A multivalent support may be protein-based, including single amino acid polymers such as polylysine, polyarginine and polyhistidine, gelatin, collagen, complex ‘natural’ protein carriers such as albumins and globulins; and including recombinant versions and combinations of these. A multivalent support may also be carbohydrate-based, including for instance polymers such as carrageen, alginates, pectins and celluloses. In addition, a multivalent support includes those that are lipid-based, such as cationic lipids of various compositions, liposome compositions of various types as described for instance in R. R. C. New, Liposomes: A Practical Approach, Oxford University Press.

A preferred multivalent support includes a dendrimer. Dendrimer creation is generally characterized by multistage synthesis of “generational” products having an organized “branched” structure. A dendrimer is synthesized from monomers added in stepwise fashion, such that each synthetic step produces a “generation” which is added to in a subsequent step. A dendrimer included as a multivalent support in an inventive composition is preferably a divergent dendrimer. Each “generation” of dendrimers has an increasing number of terminals available for conjugation to a functional group. Thus, for example, a first generation dendrimer may have 4 sites available for functional group conjugation, a second generation 8, etc. Depending on factors such as steric hindrance, the number of functional groups conjugated to the sites available for attachment on the dendrimer may be equal to or less than the maximum number of sites available on the dendrimer. Various types of dendrimers are known in the art including polyamidoamine (PAMAM) and poly (propylene imine) dendrimers. Additional dendrimer compositions may be used as a multivalent support in an inventive composition include L-lysine and N,N′-bis(acrylamido)acetic acid dendrimers. Dendrimer compositions, in the context of the present invention are advantageous due to their low immunogenicity. In addition, dendrimer compositions are stable and effectively mimic the natural clustering of lipid rafts.

A preferred inventive composition is described by the formula [(X)—(Y)]ₚ-Z. wherein X is a galactose residue including a sulfur or phosphate containing group. An inventive composition is represented in this embodiment by the formula:

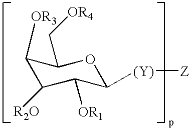

In this formula, at least one of the variables R1, R2, R3 and R4 includes a sulfur or phosphate containing group and each of the other variables R1, R2, R3 and R4 is independently H or a sulfur or phosphate containing group. The variables Y, p and Z are as described herein. A sulfur or phosphate containing group includes SO3-, SO3H, SO3D, PO3-, PO3H, or PO3D, where D is a cation. Particularly preferred is an embodiment in which X is galactose-3-sulfate, that is, in which R2 is SO3-, SO3H or SO3D, where D is a cation, and R1, R3 and R4 are H.

Optionally, in an embodiment in which p is 2-2000 in which each galactose residue includes at least one sulfur or phosphate containing group, the sulfur or phosphate containing group included in each galactose residue is independently positioned at R1, R2, R3 or R4. Thus, for example, each of the total number, p, of sulfated galactose residues is galactose-3-sulfate in a particularly preferred embodiment.

In further embodiments, each of the total number, p, of sulfated galactose residues is identically sulfated and is selected from the group consisting of galactose-2-sulfate, galactose-4-sulfate, and galactose-6-sulfate. In other embodiments, each of the total number, p, of sulfated galactose residues is identically sulfated and is selected from the group consisting of galactose-2,3-sulfate, galactose-2,4-sulfate, galactose-2,6-sulfate, galactose-3,4-sulfate, galactose-3,6-sulfate, galactose-4,6-sulfate, galactose-2,3,4-sulfate, galactose-2,3,6-sulfate, galactose-3,4,6-sulfate, galactose-2,4,6-sulfate, and galactose-2,3,4,6-sulfate.

In a further preferred embodiment, sulfation is random, such that each of the total number, p, of sulfated galactose residues is sulfated on at least one R position and each sulfated galactose residue is independently selected from the group consisting of: galactose-2-sulfate, galactose-3-sulfate, galactose-4-sulfate, and galactose-6-sulfate, galactose-2,3-sulfate, galactose-2,4-sulfate, galactose-2,6-sulfate, galactose-3,4-sulfate, galactose-3,6-sulfate, galactose-4,6-sulfate, galactose-2,3,4-sulfate, galactose-2,3,6-sulfate, galactose-3,4,6-sulfate, galactose-2,4,6-sulfate, and galactose-2,3,4,6-sulfate.

In a preferred embodiment, a inventive composition which is randomly sulfated includes an average of 1-4 sulfur containing groups per galactose residue. In a further preferred embodiment, an inventive composition includes an average of 2-3 sulfur containing groups per galactose residue. In another preferred embodiment, an inventive composition includes an average of 3-4 sulfur containing groups per galactose residue. In a preferred embodiment a sulfur or phosphate containing group is SO3- or SO3D where D is H or a cation.

In addition to sulfated compounds, a composition of the present invention includes phosphate containing compounds. In particular, an inventive compound may include PO3 or PO3D where D is H or a cation.

A preferred inventive composition is described by the formula [(X)—(Y)]ₚ-Z. wherein X is a glucose residue including a sulfur or phosphate containing group. An inventive composition is represented in this embodiment by the formula:

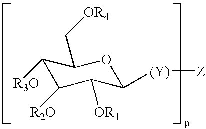

In this formula, at least one of the variables R1, R2, R3 and R4 includes a sulfur or phosphate containing group and each of the other variables R1, R2, R3 and R4 is independently H or a sulfur or phosphate containing group. The variables Y, p and Z are as described herein.

In a further preferred embodiment, X is a disaccharide residue having a sulfur or phosphate containing group. Optionally, each of the monosaccharide units included in the disaccharide includes a sulfur or phosphate containing group. A disaccharide residue included in an inventive composition preferably includes a hexose monosaccharide unit, and further preferably includes two hexose monosaccharide units. Exemplary disaccharides include a monosaccharide unit selected from the group consisting of: allose, altrose, glucose, mannose, gulose, idose, galactose, talose and combinations thereof. A preferred disaccharide includes a sulfated galactose residue. A further preferred disaccharide is a sulfated lactose residue, galactoseβ1-4glucose.

In one embodiment, an inventive composition is represented by the formula:

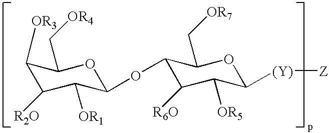

In this formula representing a composition in which X is galactoseβ1-4glucose, at least one of the variables R1, R2, R3 and R4 includes a sulfur or phosphate containing group and each of the other variables R1, R2, R3 and R4 is independently H or a sulfur or phosphate containing group. Optionally, at least one of the variables R5, R6, and R7 includes a sulfur or phosphate containing group and each of the other variables R5, R6, and R7 is independently H or a sulfur or phosphate containing group. The variables Y, p and Z are as described herein.

In a further preferred embodiment, X is a trisaccharide residue having a sulfur or phosphate containing group. Optionally, each of the monosaccharide units included in the trisaccharide includes a sulfur or phosphate containing group. A trisaccharide residue included in an inventive composition preferably includes a hexose monosaccharide unit, and further preferably includes three hexose monosaccharide units. Exemplary trisaccharides include a monosaccharide unit selected from the group consisting of: allose, altrose, glucose, mannose, gulose, idose, galactose, talose and combinations thereof. A preferred trisaccharide includes a sulfated galactose residue and particularly, a terminal sulfated galactose residue. A further preferred trisaccharide is a sulfated galactoseα1-4 galactoseβ1-4glucose as depicted in the following formula:

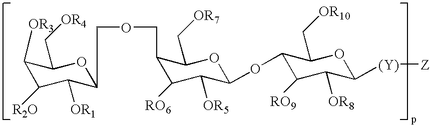

In this formula at least one of the variables R1, R2, R3 and R4 includes a sulfur or phosphate containing group and each of the other variables R1, R2, R3 and R4 is independently H or a sulfur or phosphate containing group. Optionally, at least one of the variables R5, R6, and R7 includes a sulfur or phosphate containing group and each of the other variables R5, R6, and R7 is independently H or a sulfur or phosphate containing group. In a further option, at least one of the variables R8, R9, and R10 includes a sulfur or phosphate containing group and each of the other variables R8, R9, and R10 is independently H or a sulfur or phosphate containing group. The variables Y, n, p and Z are as described herein.

Another preferred embodiment includes a composition in which X is a sulfated sialic acid(α2-3)galactose(β1-4)glucose residue.

In some embodiments an inventive composition includes X where X is a saccharide residue including 4-10 monomer saccharide units of which at least one of the 4-10 monomer saccharide units includes a sulfur or phosphate containing group. Each monomer saccharide unit is optionally selected from the group consisting of: hexose, pentose, tetrose, triose monomer saccharides. Monomer saccharides are also selected from 7-, 8-, 9-, 10-, 11-, 120 or more carbon sugars and modified sugars, such as sialic acid. Exemplary 4 monomer saccharides include sulfated galNAc(β1-4)[sialic acid(α2-3)]galactose(β1-4)glucose and sulfated sialic acid(α2-8)sialic acid(α2-3)galactose(β1-4)glucose. In a preferred embodiment, at least one monomer saccharide is a hexose saccharide, and further preferably, a sulfated galactose or glucose residue. Optionally each monomer sugar unit includes a sulfur or phosphate containing group.

In a preferred embodiment of an inventive composition, a terminal monosaccharide residue included the saccharide X is sulfated. The term terminal saccharide is used herein to describe a monosaccharide residue included in a di-, tri- or oligo-saccharide wherein the terminal monosaccharide residue is the most distal, relative to Z, of the monosaccharide residues included in X. Where X is a branched tri- or oligo-saccharide, more than one terminal monosaccharide is present. In the case that X is a linear tri- or oligo saccharide, a single terminal monosaccharide is present.

A composition according to the invention includes a compound represented by the formula:

[(X)—(Y)]ₚ-Z

where X is

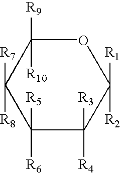

where R1, R2, R3, R4, R5, R6, R7, R8, R9 and R10 are each independently H, OH, COOH, sialic acid, a saccharyl group, OSO3D, OPO3D, a bond to Y, or a bond to Z, CH2OH, CH2OSO3D, or CH2OPO3D, where D is H or a cation selected from the group consisting of: alkali metal cations, alkaline earth metal cations, ammonium cations, quaternary ammonium cations and amine cations; where at least one of R1, R2, R3, R4, R5, R6, R7, R8, R9 and R10 is a bond to Y or a bond to Z; where the bond to Y is a bond to an atom selected from the group consisting of: S, O, P, N, or C; where at least one of R1, R2, R3, R4, R5, R6, R7, R8, R9 and R10 has a sulfur or phosphate containing group; and where Y is an optional linker, Z is a multivalent support, and p is an integer in the range of 1-2000.

Where one or more of the variables R1, R2, R3, R4, R5, R6, R7, R8, R9 or R10 is a saccharyl group, a saccharyl group has the formula:

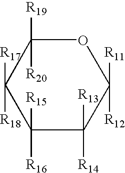

where R11, R12, R13, R14, R15, R16, R17, R18, R19 and R20 are each independently H; OH; COOH; sialic acid; a saccharyl group; OSO3D; OPO3D; a bond to R1, R2, R3, R4, R5, R6, R7, R8, R9 or R10; CH2OH; CH2OSO3D; or CH2OPO3D; where D is H or a cation selected from the group consisting of: alkali metal cations, alkaline earth metal cations, ammonium cations, quaternary ammonium cations and amine cations; where at least one of R11, R12, R13, R14, R15, R16, R17, R18, R19 and R20 is a bond to R1, R2, R3, R4, R5, R6, R7, R8, R9 or R10; and where the bond to R1, R2, R3, R4, R5, R6, R7, R8, R9 or R10 is a bond to an atom selected from the group consisting of: S, O, P, N, or C.

The term saccharyl group is used to indicate a substituent of a saccharide residue X. A saccharyl group is itself a saccharide residue as described herein with the exception that a saccharyl group does not contain a bond to Y or Z. Optionally, a saccharyl group is a monosaccharide residue as described herein. Further optionally, a saccharyl group is a disaccharide residue as described herein. In an additional option, a saccharyl group is a trisaccharide residue as described herein. In some embodiments, an optional saccharyl group has 4-10 monosaccharide units. In further embodiments, an optional saccharyl group has 10-50 monosaccharide units. Each individual monosaccharide unit optionally includes sulfur and/or phosphate containing groups.

Thus, in an embodiment of the invention, a saccharyl group is a monosaccharide substituent of another monosaccharide and thus X is a disaccharide. In a further embodiment of the invention, a saccharyl group is a disaccharide substituent of another monosaccharide and X is a trisaccharide. In another embodiment, a saccharyl group is a trisaccharide and X is an oligomer including 4 monosaccharide residues. As noted, a bond between X and a saccharyl group is selected from S, O, P, N, or C. Particularly preferred is a configuration in which a bond between X and a saccharyl group is O or S. In some embodiments a bond between X and a saccharyl group is preferably S, a configuration resistant to O-glycosidases. Optionally a bond between monosaccharides of a saccharyl group is selected from S, O, P, N, or C. In a preferred option, the monosaccharides forming a disaccharide, trisaccharide or 4-10 monosaccharide saccharyl group are bonded by O- or S-glycosidic linkage.

Further optionally, each of the monosaccharide of a saccharyl group independently includes a sulfur or phosphate containing group as described herein.

And example of such a compound [(X)—(Y)]ₚ-Z is a sulfated sialic acid(α2-3)galactose(β1-4)glucosyl thiopropionic acid conjugated to a dendrimer. In that case X may be described as a glucose residue having a saccharyl group, sialic acid(α2-3)galactose in β1-4 linkage with the glucose residue.

In a further embodiment, an inventive composition has the formula: [(X)—(Y)]ₚ-Z, where X is

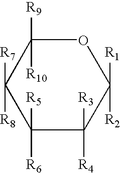

where R1, R2, R3, R4, R5, R6, R7, R8, R9 and R10 are each independently H, OH, COOH, sialic acid, a saccharyl group, NHAc, C1-8 acyl, anhydro, C1-8 alkyl, NH2, halogen, OSO3D, OPO3D, a bond to Y, a bond to Z, CH2OH, CH2OSO3D, or CH2OPO3D; where D is H or a cation selected from the group consisting of: alkali metal cations, alkaline earth metal cations, ammonium cations, quaternary ammonium cations and amine cations; where at least one of R1, R2, R3, R4, R5, R6, R7, R8, R9 and R10 is a bond to Y or a bond to Z; where the bond to Y is a bond to an atom selected from the group consisting of: S, O, P, N, or C; where at least one of R1, R2, R3, R4, R5, R6, R7, R8, R9 and R10 has a sulfur or phosphate containing group; and where Y is an optional linker, Z is a multivalent support, and p is an integer in the range of 1-2000.

An optional saccharyl group has the formula:

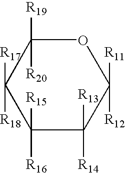

where R11, R12, R13, R14, R15, R16, R17, R18, R19 and R20 are each independently H, OH, COOH, sialic acid, a saccharyl group, NHAc, C1-8 acyl, anhydro, C1-8 alkyl, NH2, halogen, OSO3D, OPO3D, a bond to R1, R2, R3, R4, R5, R6, R7, R8, R9 or R10, CH2OH, CH2OSO3D, or CH2OPO3D, where D is H or a cation selected from the group consisting of: alkali metal cations, alkaline earth metal cations, ammonium cations, quaternary ammonium cations and amine cations; where at least one of R11, R12, R13, R14, R15, R16, R17, R18, R19 and R20 is a bond to R1, R2, R3, R4, R5, R6, R7, R8, R9 or R10; where the bond to R1, R2, R3, R4, R5, R6, R7, R8, R9 or R10 is a bond to an atom selected from the group consisting of: S, O, P, N, or C.

A saccharyl group is, for instance, a monosaccharide, disaccharide or trisaccharide. Optionally, a saccharyl group is a saccharide residue having 4-10 monosaccharide units.

A pharmaceutical composition according to the invention includes an inventive compound as described herein, and may further contain a pharmaceutically acceptable carrier or excipient formulation. The term “pharmaceutically acceptable” is intended to mean a material that is not biologically or otherwise undesirable, which can be administered to an individual along with an inventive compound without causing significant undesirable biological effects or interacting in a deleterious manner with any of the other components of the pharmaceutical composition in which it is contained. A pharmaceutically acceptable carrier or excipient formulation may include sterile aqueous or non-aqueous solutions, dispersions, suspensions or emulsions, and sterile powders for reconstitution into sterile administrable solutions or dispersions. Examples of suitable aqueous and non-aqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

Additionally, a pharmaceutical composition according to the invention may include other pharmaceutical agents. The general content of a pharmaceutically acceptable carrier or excipient formulation will depend on the form in which the pharmaceutical composition containing an inventive compound is given. For instance, depending on the intended mode of administration, a pharmaceutical composition containing an inventive compound is delivered in solid, semi-solid or liquid dosage forms, such as, for example, tablets, suppositories, pills, capsules, powders, liquids, or suspensions, preferably in unit dosage form suitable for single administration of a precise dosage.

An inventive composition may be administered orally, parenterally (for example, intravenously), by intramuscular injection, by intraperitoneal injection, or transdermally. For oral administration, fine powders or granules may contain diluting, dispersing, and/or surface active agents, and may be presented in water or in a syrup, in capsules or sachets in the dry state or in a nonaqueous solution or suspension wherein suspending agents may be included, in tablets wherein binders and lubricants may be included, or in a suspension in a liquid or gel. Tablets and granules are preferred oral administration forms, and these may be coated. Parenteral administration is generally by injection. Injectables can be prepared in conventional forms, either liquid solutions or suspensions, solid forms suitable for solution or prior to injection, or as suspension in liquid prior to injection or as emulsions.

For solid compositions, an excipient formulation may include conventional nontoxic solid carriers or fillers include, for example, pharmaceutical grades of cellulose, glucose, lactose, magnesium carbonate, magnesium stearate, mannitol, sodium saccharine, silicic acid, starches, sucrose and talc.

Liquid pharmaceutically administrable compositions can, for example, be prepared by dissolving or dispersing an inventive compound with optional pharmaceutical adjuvants in an excipient or inert diluent to thereby form a solution or suspension. Liquid dosage forms include pharmaceutically acceptable emulsions, solutions, suspensions, syrups, and elixirs. Thus, in addition to the active compounds, a liquid dosage form may contain such excipients or inert diluents as water or other solvents, solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances, and the like.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

An excipient formulation may contain further inert customary ingredients, such as binders, as for example, carboxymethylcellulose, alignates, gelatin, polyvinylpyrrolidone, sucrose, and acacia; humectants, as for example, glycerol; disintegrating agents, as for example, agar-agar, calcium carbonate, potato or tapioca starch, alginic acid, certain complex silicates, and sodium carbonate; solution retarders, as for example paraffin; absorption accelerators, as for example, quaternary ammonium compounds; wetting agents, as for example, cetyl alcohol, and glycerol monostearate; adsorbents, as for example, kaolin and bentonite; and lubricants illustratively including talc, calcium stearate, magnesium stearate, solid polyethylene glycols, sodium lauryl sulfate, or mixtures thereof. Besides such ingredients, the excipient formulation can also include adjuvants, such as emulsifying agents, sweetening, flavoring, and perfuming agents.

Where continuous pharmaceutical composition delivery is required, time release preparations or intravenous preparations are exemplary effective dosage formulations. Illustrative examples of release control components include: carbomer, alpha-starch, polyacrylamides, polysaccharides, polyvinylpyrrolidone; natural gums such as gum arabic; clays; lipophilic gelling agents; fatty acid metal salts such as aluminum stearates; hydrophobic silica; celluloses such as various hydroxyalkylcelluloses; polyethylene glycol; and combinations thereof.

If desired, the pharmaceutical composition to be administered may also contain minor amounts of nontoxic auxiliary substances such as pH buffering agents, for example, sodium acetate, sodium citrate, dicalcium phosphate or triethanolamine oleate. Contamination by microorganisms can be prevented or inhibited by various antibacterial and antifungal agents, for example, parabens, chlorobutanol, phenol, sorbic acid, and the like. It may also be desirable to include isotonic agents, for example sugars, sodium chloride, and the like. Actual methods of preparing such dosage forms are known, or will be apparent, to those skilled in this art; for example, see A. Gennaro, Remington: The Science and Practice of Pharmacy, 2000, Lippincott, Williams & Wilkins; Goodman and Gilman's The Pharmacological Basis of Therapeutics by Hardman and Limbird, 9th Ed., 1996, McGraw-Hill, New York and in The Merck Index: an encyclopedia of chemicals, drugs, and biologicals, 12th Edition, 1996, Merck & Co., Whitehouse Station, N.J.

The exact amount of an inventive pharmaceutical composition required will vary from subject to subject, depending on the age, weight and general condition of the subject, the condition being treated, the particular compound used, its mode of administration, and the like. For example, formula of Freireich et al., Cancer Chemother. Rep., 50:219-244, (1966) can be used to determine the maximum tolerated dose of an inventive compound for a human subject. An appropriate amount may be determined by one of ordinary skill in the art using only routine experimentation given the teachings herein.

A process according to the present invention is provided for inhibiting interaction of a ligand and a receptor. An inventive process includes the step of providing a composition as described herein. The composition is provided as an inventive compound or as a pharmaceutical composition as described herein.

The term “ligand” as used herein is intended to mean a cell, organism, molecule, or a molecular group that interacts with another entity, identified by the term “receptor”, wherein a receptor is a cell, organism, molecule, or a molecular group. The term “interaction” as used herein is intended to mean contact which produces a specific effect or cascade of effects. Specifically contemplated as within the meaning of a ligand/receptor interaction which produces a specific effect or cascade of effects is the interaction of an organism and a cell wherein the interaction of the organism and cell results in a specific effect—for instance, infection of the cell or, more generally, infection of an animal of which the cell is a part. Thus, for example, in one embodiment of an inventive process, interaction of a virus and a cell susceptible to viral infection is an interaction of a ligand and receptor within the meaning given to those terms in the instant application. It will be evident to one of skill in the art that specific interaction of a virus and a cell susceptible to infection by the virus may be mediated by one or more specific molecular ligands present on or emitted by the virus and/or one or more specific molecular receptors present on or emitted by the cell, to result in the infection. Thus, in describing the interaction of a ligand and receptor where the ligand is a virus and the receptor is a cell, the ligand and receptor may also be described in terms of their molecular identity, when known. For example, a ligand or receptor may be a particular protein expressed by a virus or cell. Thus, the ligand or receptor may be a virus or component of a virus. Further the ligand or receptor may be a cell or a component of a cell. In a preferred embodiment, the virus is a virus of the family Retroviridae. In a further preferred embodiment the virus is a lentivirus, including HTLV-1, HTLV-II, non-human associated lentivirus including SIV, avian myeloblastosis virus and leukosis virus, feline leukemia virus and feline immunodeficiency virus. Further included is a chimeric virus such as SHIV. A preferred virus is a human immunodeficiency virus, such as HIV-1 and HIV-2. A component of a virus which interacts with a receptor is a gpl20 protein of an HIV. A cell which is susceptible to infection by a virus is an animal cell, in vitro or in vivo. In particular, cells susceptible to infection by a retrovirus, especially HIV, are receptors within the meaning of the present application, where a retrovirus, especially HIV, is a ligand. Such cells include human T cells.

Optionally, the ligand is a mycoplasma or component of a mycoplasma and the associated receptor is a retrovirus, particularly HIV.

Optionally, interaction of a first cell and a second cell is an interaction of a ligand and receptor within the meaning given to those terms in the instant application. In particular, a specifically contemplated embodiment of an inventive process includes interaction of a first cell wherein the first cell is a sperm cell and a second cell wherein the second cell is an ovum. Optionally, the sperm and ovum are human cells. In this embodiment, a specific effect of the interaction of the first and second cells is fertilization of the ovum. Again, it will be evident to one of skill in the art that specific interaction of a first cell and a second cell may be mediated by one or more specific molecular ligands present on or emitted by the first cell and/or one or more specific molecular receptors present on or emitted by the second cell. Thus, the ligand and/or receptor may be a cell or a component of a cell.

Further exemplary ligand/receptor pairs include cell-cell, wherein a first cell is a normal cell and a second cell is a pathological cell, cell-organism such as wherein a first cell is a vertebrate cell and a second cell is a bacterial cell, protein-protein such as antigen-antibody, binding protein-toxin, substrate-enzyme, effector-enzyme, inhibitor-enzyme, complimentary nucleic acid strands, binding protein-vitamin, binding protein-nucleic acid, reactive dye-protein, and reactive dye-nucleic acid interactions and carbohydrate-lectin.

A further step of an inventive process includes introducing the composition into an environment containing the ligand or the receptor. In a preferred embodiment, the environment is a human or non-human animal body. Thus, for example, where the ligand is a virus and the receptor is a cell, an inventive composition is placed in the vicinity of the receptor. For instance, where the receptor is a circulating T cell, the composition is advantageously administered systemically, especially intravenously. Further, an inventive composition may be placed at sites of likely admission of a ligand virus to the body. For example, the composition is advantageously placed at oral, anal and vaginal sites. In addition, the composition is placed at wound sites, sites vulnerable to trauma, and sites vulnerable to contact with substances likely to contain virus.

Optionally, the environment is an in vitro environment. For example, an inventive compound or composition may be a component of an assay for evaluation of drug candidates. Further, an inventive compound or composition may be a component of an in vitro assay to characterize a virus or receptor, for example, to characterize infection capabilities of a novel virus strain.

An inventive process of therapeutic or prophylactic anti-viral treatment includes the step of administering an effective amount of an inventive compound or composition to an individual human or non-human animal in need of such therapeutic or prophylactic anti-viral treatment. In particular, an inventive compound or composition is administered to an individual human or non-human animal at risk of retroviral infection or in need of treatment for retrovirus infection. In a preferred embodiment, an inventive compound or composition is administered to an individual human or non-human animal at risk of HIV infection or in need of treatment for HIV infection.

Optionally, a compound or composition according to the invention is administered to inhibit ligand/receptor interaction along with a second composition. In particular, an inventive compound or composition is administered to an individual subject in order to inhibit interaction of an HIV virus with a cell or component of a cell in the individual subject along with another composition, such as a protease inhibitor, nucleoside analog, virucidal agent, vaccine or antibody, anti-viral nucleic acid such as an anti-sense construct or SIRNA, immune modulator, zinc finger inhibitor, integrase inhibitor or TAT inhibitor.

An inventive compound or composition is included in a commercial package, for instance for use in an in vitro or in vivo application, along with instructions for use.

### EXAMPLES

In describing methods and compounds according to the present invention, abbreviations may be used. The following are terms which are used in abbreviated form along with the corresponding unabbreviated term: Bu2SnO, dibutyltin oxide; BF3Et2O, borontriflouride dietherate; COSY, correlated spectroscopy; DCM, dichloromethane; DCE, dichloroethane; DIPEA, diisopropylethylamine; DxS, dextran sulfate; DAB-Am, poly(propylene imine) dendrimers; DMF, dimethyl formamide; Et3N, triethylamine; GalCer, galactosyl ceramide; HATU, N,N,N′,N′-tetramethyl-O-(7-azabenzotriazol-1-yl)uronium hexafluorophosphate; HMBC, heteronuclear multiple bond correlation; HMQC, heteronuclear multiple quantum correlation; HPTLC, high performance thin layer chromatography; IAA, trans-3-indoleacrylic acid; KMnO4, potassium permangenate; ka, association rate constant; kd, dissociation rate constant; KD, equilibrium dissociation constant; kDa, kilo Daltons; Mn, number average molecular weight; Mw, weight-average molecular weight; MALDI-TOF MS, matrix-assisted laser desorption ionization time-of-flight mass spectrometry; MTS 3-(4,5-dimethylthiazol-2-yl)-5-(3-carboxymethoxyphenyl)-2-(4-sulfophenyl)-2H-tetrazolium, inner salt; NaOMe, sodium methoxide; PD, polydispersity; PMS, phenazine methosulfate; SO3NMe3, sulfur trioxide trimethylamine complex; SGalCer, sulfatide; SPR, surface plasmon resonance; Rmax, maximum binding capacity in RUs; RU, resonance unit (change in surface concentration of ˜1 pg of protein/mm2); THAP, trihydroxyacetophenone.

### Example 1

### Materials

GalCer, SGalCer, lyso-sulfatide, and psychosine standards are purchased from Matreya, Inc (Pleasant Gap, Pa.). GalCer and SGalCer for ceramide saccharide syntheses are purified from bovine brain white matter as described in Schengrund, C. L. and Ringler, N. J., 1989, J Biol Chem 264, 13233-13237. Recombinant HIV-1 IIIB gpl20 is obtained from Immunodiagnostics, Inc. rgpl20 MN is obtained from ImmunoDiagnostics through the National Institutes of Health (NIH) AIDS Reagent Program, and rgpl20 Ba-L is from Dr. Raymond Sweet (SmithKline Beecham, King of Prussia, Pa.). Human polyclonal anti-HIV IgG antibody is also from the AIDS Reagent Program. Direct-pelleted HIV-1 viruses IIIB, MN, Ba-L, and 89.6 are obtained from ABI (Columbia, Md.). Dextran sulfate (DxS) (8 kDa and 50 kDa), chondroitin sulfate (ChS) (45 kDa), glucose-3-sulfate, galactose-6-sulfate, galactose-4-sulfate, and sialyllactose are from Sigma (St. Louis, Miss.). Galactose-3-sulfate is isolated from SGalCer as described in Miljkovic, M., and C. L. Schengrund, 1986, Carbohydr Res. 155:175-81; and Yowler, B. C., et al., 2001, J Lipid Res. 42:659-62. DAB-Am dendrimers from Aldrich (Milwaukee, Wiss.), and silica gel 60 HPTLC plates are from Merck (Germany). Unless designated otherwise, all other materials are commercially available, and used without further purification.

### Preparation of Peracetylated GalCer Ceramide Saccharide

Peracetylated GalCer ceramide saccharide, is prepared according to a process depicted in Scheme 1 below.

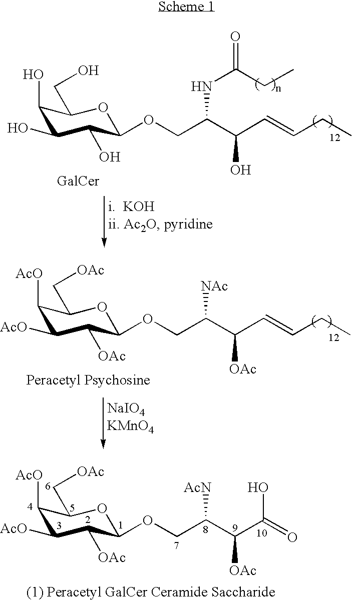

In order to build multivalent GalCer and SGalCer derivatized dendrimers, the saccharide moiety of GalCer and SGalCer (galactose and galactose-3-sulfate, respectively), containing a portion of the sphingosine moiety, were isolated from the natural GSLs (Scheme 1). GalCer is deacylated to form psychosine using reaction conditions described in Radin, N. S., 1974, Lipids 9, 358-360. and in Dubois, G., et al., 1980, Anal Biochem 102, 313-317, for the removal of the fatty acid side-chain from GSLs. The ceramide saccharide derivative of GalCer is made from psychosine according to procedures described in Mylvaganam, M. and Lingwood, C. A., 2000, Methods Enzymol 312, 473-487. MALDI-TOF MS m/z 548 [M−H]−: C22H31NO15, peracetylated GalCer ceramide saccharide (549). Techniques described by Mylvaganum and Lingwood (Id.), for the preparation of glycolipid ceramide saccharides for neoglycoconjugation were used for the synthesis of the peracetylated ceramide saccharides directly from GalCer (1) and SGalCer. This is achieved by deacylation of the GSL followed by its acetylation to block vicinal hydroxyls prior to oxidative cleavage of the double bond of the sphingosine to obtain the acetylated “ceramide saccharide” (Scheme 1). The carboxyl group of the N-acetylated butyric acid provided the reactive group needed to link the sugar to a primary amine on the dendrimer core. The yield of ceramide saccharide derivative of SGalCer obtained is less than 20%. According to MALDI-TOF MS, deacetylation of the peracetyl SGalCer ceramide saccharide, resulted in the loss of approximately 40% of the sulfate residues. Note that the numbering system for NMR analysis is shown in scheme 1.

### Example 2

### Preparation of 3-(β-D-galactopyranosylthio)propionic acid (Identified as 2 in Scheme 2)

The method described by Ashton, et al., 1997, Chem. Eur. J. 3, 974-984, for the synthesis of 3-(β-D-galactopyranosylthio)propionic acid (2) is used to derivatize the sugars with a spacer arm that could be used to link them to the DAB-Am dendrimers. This method is efficient, with the yield of derivatized galactose molecules greater than 80%. While 3-(β-D-galactopyranosylthio)propionic acid (2) is not made directly from natural GSLs, its structure is similar to structure 1 shown in Scheme 1. Further, 3-(β-D-galactopyranosylthio)propionic acid (2) is advantageous in synthesis of neoglycoconjugates since the thioglycosidic linkage is more resistant to cleavage by endogenous glycosidases than the corresponding O-glycosides, see for example, Driguez, H., 1997, Thiooligosaccharides in glycobiology. Topics Curr. Chem. 187, 85-116.

To prepare 3-(β-D-galactopyranosylthio)propionic acid, 3-mercaptopropionic acid (10.5 mL, 120 mmol) is added with stirring to a solution of galactose-β-pentaacetate, 11.7 g (30 mmol), in 60 mL anhydrous DCM. This is followed by the addition of 5.7 mL (45 mmol) BF3.Et2O. See Manusson G, N. G., et al., 1981, Acta Chemica Scandinavica B 35, 213-216 and Elofsson M, W. B., Kihlberg J., 1991, Tetrahedron Letters 32, 7613-7616 for further details. The reaction is stirred at room temp and followed by TLC (BuOH:CH3OH:H2O; 2:1:1, v/v/v). Upon completion (˜5 hrs), the reaction mixture is poured into ice water in a separatory funnel. The lower organic phase is removed and the aqueous phase is rinsed 3× with DCM. The combined organic phases are washed with water, treated with anhydrous sodium sulfite, filtered over Celite, and dried by rotary evaporation. Peracetylated galactothiopropionic acid is isolated from the resulting viscous oil on a silica gel column, eluted with a step gradient of DCM:CH3OH of increasing polarity. It is recovered as a colorless oil in 80% yield. For NMR analysis and regioselective sulfation, a portion of the peracetylated galactothiopropionic acid is deacetylated using NaOMe in anhydrous CH3OH. After stirring overnight at room temp, it is neutralized using DOWEX 50W [H+]. 1H NMR (400 MHz, D2O, 25° C.): δ=2.50 (t, J7A,8≈7 Hz, J7B,8≈7 Hz, 2H, H-8), 2.90 (m, J7A,8≈7 Hz, J7B,8≈7 Hz, 2H, H-7), 3.52 (t, J1,2=9.72 Hz, J2,3=9.6 Hz,1H, H-2), 3.61 (dd, J2,3=9.6 Hz, J3,4=3.4 Hz,1H, H-3),3.66 (m, 1H, H-6B), 3.67 (m, J4,5=0.2 Hz,1H, H-5), 3.72 (m, 1H, H-6A), 3.93 (d, J3,4=3.36 Hz, J4,5=0.2 Hz, 1H, H-4), 4.46 (d, J1,2=9.72 Hz, 1H, H-1). 13C NMR (D2O, 25° C.): δ=27.3 (C-7), 38.6 (C-8), 61.7 (C-6), 69.5 (C-4), 70.3 (C-2), 74.55 (C-3), 79.6 (C-5), 86.65 (C-1), 181.4 (C-9). MALDI-TOF MS m/z 291 [M+Na]+: C9H16O7S (268). The numbering system for NMR analysis is shown in Scheme 2 below.

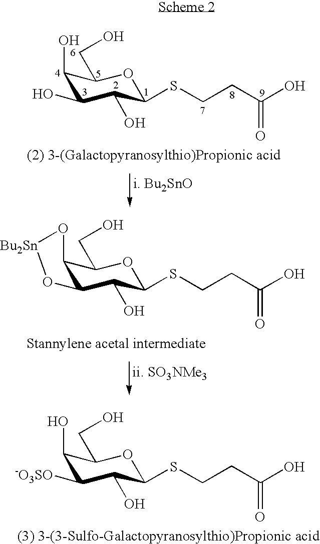

### Example 3

### Preparation of 3-(3-sulfo-β-D-galactopyranosylthio)propionic acid (Identified as 3 in Scheme 2)

In order to make a spacer-arm derivatized galactose-3-sulfate molecule, the novel 3′ sulfated derivative of 3-(β-D-galactopyranosylthio)propionic acid, is made according to methods described by Guilbert B, D. N., et al., 1994, Tetrahedron: Asymmetry 5, 2163-2178 and Lubineau, A., and Lemoine, R., 1994, Tetrahedron Letters 35, 8795-8796 for the regioselective sulfation of dibutylstannylene acetals. In this technique, dibutyltin oxide is used to form stannylene acetal complexes between cis diols, C-3 and C-4 on compound 2 as shown in Scheme 2. Further details of this step are described in David, S., and Hanessian, S., 1985, Tetrahedron 41, 643-663. The C-3 was then preferentially sulfated by reacting the complex with SO3NMe3 in DMF for 30 hrs at 60° C. After purification, 3-(3-sulfo-β-D-galactopyranosylthio)propionic acid is obtained in ˜70% yield. A specific procedure for this synthesis employs galactothiopropionic acid, 5.86 g (21.87 mmol), and Bu2SnO, 5.99 g (1.1 eq, 24.05 mmol) which are refluxed in 200 mL anhydrous CH3OH for 4 h at 90° C. (54,55). The resulting translucent yellow solution is dried by rotary evaporation, taken up in 150 mL DMF, and 3.42 g SO3NMe3 (1.2 eq, 24.6 mmol) is added. The mixture is sonicated (˜1 min), and stirred at room temp for 30 h. See Guilbert B, D. N., et al., 1994, Tetrahedron: Asymmetry 5, 2163-2178 and Nishida, Y., et al., 2000, Biomacromolecules 1, 68-74 for further details. The reaction is quenched with CH3OH, dried by rotary evaporation using an oil vacuum pump, and purified on a silica gel column eluted with a CHCl3:CH3OH step gradient. 1H NMR (400 MHz, D2O, 25° C.): δ=2.68 (t, J7A,8≈7 Hz, J7B,8≈7 Hz, 2H, H-8), 2.97 (m, J7A,8≈7 Hz, J7B,8≈7 Hz, 2H, H-7), 3.70 (dd, J1,2=9.90 Hz,1H, H-2), 3.72 (m, 1H, H-6B), 3.75 (m, 1H, H-5), 3.75 (m, 1H, H-6A), 4.32 (s, 1H, H-4), 4.33 (dd, 1H, H-3), 4.60 (d, J1,2=9.90 Hz, 1H, H-1). 13C NMR (D2O, 25° C.): δ=26.4 (C-7), 36.9 (C-8), 61.6 (C-6), 67.9 (C-4), 68.3 (C-2), 79.25 (C-5), 82.1 (C-3), 86.3 (C-1), 179.0 (C-9). MALDI-TOF MS m/z 347 [M]−: C9H15O10S2-(347). The numbering system for NMR analysis is shown in Scheme 2 above. NMR data confirms that sulfation occurred at position C-3. The chemical shifts obtained agree with those published by Ikami, et al. (1997) for compounds they synthesized as sulfatide mimetics (64). HPTLC indicates that the precursors used and the compounds synthesized, identified as 1, 2, and 3 as shown in Schemes 1 and 2, contain no visible contaminants.

### Example 4

### Conjugation of Derivatized Saccharides to Polypropylenimine Dendrimers

Compounds 1, 2, and 3 as shown in Schemes 1 and 2 are conjugated to DAB-Am dendrimers, generations 1-5, by amine coupling the terminal carboxyl groups on the derivatized saccharides to the terminal primary amines on the DAB-Am dendrimers.

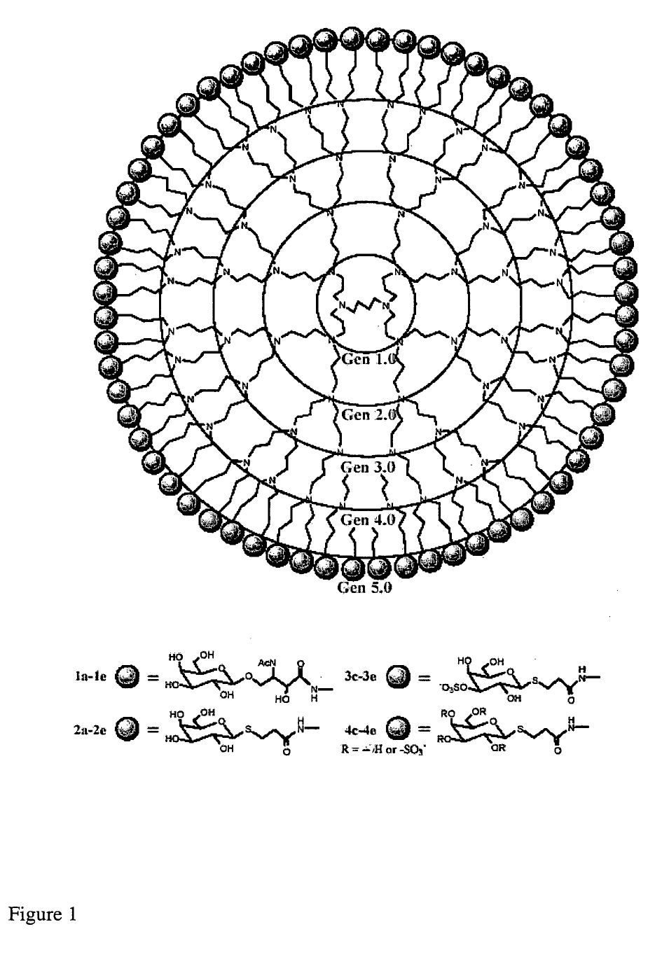

Briefly, the peracetylated derivative of compound 1, 2, or 3 as depicted in Schemes 1 and 2 and as described above, 50 to 100 mg, (1.5 equivalents per dendrimer terminal primary amine), is taken up in CH3CN (10 mg/mL). One equivalent of HATU (Aldrich, Milwaukee, Wiss.) in CH3CN (10 mg/mL) and 1 eq of DIPEA are added to each sample to activate the terminal carboxyl group as described in Poirot, E., et al., 2001, Carbohydr Res 330, 7-20. After 5-10 min, the appropriate amount of DAB-AM dendrimer at a concentration of 10 mg/ml in DCM is added dropwise to the stirring mixture. Reactions are adjusted to 1:1, DCM:CH3CN, and allowed to incubate at room temperature with stirring over night. Product formation is monitored by TLC (BuOH:CH3OH:H2O; 2:1:1, v/v/v). Free amino groups are visualized using ninhydrin spray and sugar residues using 5% sulfuric acid in ethanol. Upon completion, the mixture is dried under vacuo and the product deacetylated with Et3N:CH3OH:H2O (2:6:10; v/v/v) at a final concentration of 0.5 mg/mL. After stirring the reaction at 37° C. for 4 hr, the reaction is dried, washed with 10 mM HCl in 90% EtOH according to Mylvaganam, M. and Lingwood, C. A., Id, dried again, and purified on a BioGel P2 column. Fractions are spotted on TLC plates and carbohydrate-containing conjugates are visualized with 5% H2SO4 in EtOH. Fractions containing the purified glycodendrimers are pooled, dried, and lyophilized. Once dried, the average molecular weights of the glycodendrimers are determined by MALDI-TOF MS. In this manner glycodendrimers 1a-1e, 2a-2e, and 3c-3e are synthesized; where the designations a-e correspond to DAB-Am dendrimer generations 1-5, respectively as shown in FIG. 1. Specifically, for example, glycodendrimers that are built by conjugating species 1 depicted in Scheme 1 to DAB-Am dendrimers, generations 1-5, are referred to as glycodendrimers, 1a-1e, where 1a is the 4-mer glycodendrimer and 1e is the 64-mer. Likewise, glycodendrimers made from compounds 2 and 3, depicted in Scheme 2, are referred to as glycodendrimers 2a-2e and 3c-3e.

MALDI-TOF MS is used to determine the average number of saccharide residues incorporated onto the DAB-Am dendrimers. To calculate the average number of residues incorporated, the theoretical molecular weight of the starting DAB-Am dendrimer is subtracted from the Mw of the functionalized dendrimer and the difference divided by the weight of one derivatized saccharide, minus18 Da to account for the loss of a H2O molecule during formation of the amide bond. Table 1 shows the average molecular weights (Mw), polydispersities (PD) and average number of incorporated sugars for glycodendrimers 1a-1e, 2a-2e, and 3c-3e.

TABLE 1

| | Theoretical | | | | Avg # | Avg # | Theoretical % |
|---|---|---|---|---|---|---|---|
| Compound | Molecular Wt. | M_{N} | M_{W} | PD | sugars | sulfates | Incorporation |
| 1a GC-4mer | 1600 | 1454 | 1478 | 1.02 | 3.6 | — | 90% |
| 1b GC-8mer | 3341 | 3073 | 3100 | 1.01 | 7.2 | — | 90% |
| 1c GC-16mer | 6823 | 4978 | 5189 | 1.04 | 10.9 | — | 68% |
| 1d GC-32mer | 13,786 | 10,138 | 10,342 | 1.02 | 21.2 | — | 66% |
| 1e GC-64mer | 27,712 | 20,508 | 20,778 | 1.01 | 42.4 | — | 66% |
| 2a Gal-4mer | 1316 | 1341 | 1342 | 1.00 | 4.0 | — | 100% |
| 2b Gal-8mer | 2773 | 2634 | 2642 | 1.00 | 7.5 | — | 94% |
| 2c Gal-16mer | 5687 | 5043 | 5062 | 1.00 | 13.5 | — | 84% |
| 2d Gal-32mer | 11,514 | 9722 | 9761 | 1.00 | 25.0 | — | 78% |
| 2e Gal-64mer | 23,168 | 19,031 | 19,143 | 1.01 | 47.9 | — | 75% |
| 3c 3-sulfo-16mer | 6951 | 3867 | 4004 | 1.04 | 7.0 | 5.7 | 44% |
| 3d 3-sulfo-32mer | 14,042 | 5989 | 6184 | 1.03 | 8.1 | 6.5 | 25% |
| 3e 3-sulfo-64mer | 28,224 | 10,767 | 10,954 | 1.02 | 11.5 | 9.3 | 18% |
| 4c SGal-16mer | — | 6938 | 9010 | 1.01 | 13.5 | 12.2 | — |
| 4d SGal-32mer | — | 13,336 | 13,484 | 1.01 | 25.0 | 23.3 | — |
| 4e SGal-64mer | — | 19,425 | 24,581 | 1.01 | 47.9 | 34.2 | — |

Specific structures associated with glycodendrimers designated 1a-1e, 2a-2e, 3c-3e, 4c-4e and PS Gal, a more highly sulfated version of 4, are depicted in FIG. 1.

### Example 5

### Direct Sulfation of Dendrimers Derivatized with 3-(β-D-galactopyranosylthio) Propionic Acid

Random sulfation of glycodendrimers 2c, 2d, and 2e is done in order to make sulfated 3-(β-D-galactopyranosylthio) propionic acid-derivatized dendrimers. Glycodendrimers 2c, 2d, and 2e were randomly sulfated using a 2-fold excess of SO3NMe3 per hydroxyl group. Briefly described, in this procedure 30 mgs of 2c, 2d, or 2e are taken up in 30 mls of DMF, prior to the addition of SO3NMe3 (2 eq. per hydroxyl group). The reaction mixture is refluxed with stirring at 60° C. for 30 hrs, and then dried by rotary evaporation under reduced pressure supplied by an oil vacuum pump. Dried films were taken up in 2 mls of 1 M NaCl. The resulting randomly sulfated glycodendrimers, designated 4c, 4d, and 4e, are purified by size-exclusion chromatography on a BioGel P2 column, using water as the eluate. This gives the sulfated galactose-derivatized dendrimers 4c, 4d, and 4e as depicted in FIG. 1. Subtraction of the starting molecular weights of 2c, 2d, and 2e from the respective sulfated products 4c, 4d, and 4e, followed by division of the molecular weight of one sulfatepyridine salt minus 1 H+ (158), gives the average number of sulfate groups incorporated (Table 1).

MALDI-TOF MS analysis indicates that on average 12, 23 and 34 sulfate groups are added to the glycodendrimers 4c, 4d, and 4e, respectively (Table 1).

### Example 6

### Synthesis of Randomly Poly-Sulfated Galactose Functionalized, Generation 5.0, Glycodendrimers (PS Gal 64mer)

Generation 5.0 DAB dendrimers (64 terminal amines) are functionalized with 3-(galactosylthio)propionic acid residues as described above. and are referred to here as 2e or Gal 64mer. Gal 64mer, 60 mgs, containing on average 44 galactose residues with an Mw of 18,261 was taken up in 2 mls of dry DMSO. Sulfur trioxide pyridine complex, 2 eq. per hydroxyl, was then added, and the mixture stirred for 5 hrs at 37° C. (52). After the incubation period, pyridine was added to the mixture to yield a 1.0 M pyridine mixture that was then applied directly to a Biogel P2 column and eluted with 1.0 M pyridine acetate buffer, pH 5.0. Aliquots of fractions eluted in the void volume were spotted on TLC plates and the presence of carbohydrate-containing compounds identified by charring with 5% H2SO4 in EtOH. Carbohydrate-containing fractions were pooled and dried several times from toluene by rotary evaporation. The resulting film was taken up in 2 mls of H2O and passed through a small amount of Amberlite (Na+; 100-200 mesh) resin to convert the SO3-pyridine salt to the Na+ salt form. The resin was washed with H2O, the eluate dried by rotary evaporation, and the resulting film lyophilized. MALDI-TOF MS analysis of the product revealed that the resulting compound had a polydispersity of 1.02 and a Mw of 26,789; which corresponded to an average of 84 sulfate groups incorporated when the Mw of the galactosylated dendrimer was subtracted from that of the sulfated product, and the difference divided by 102 Da (the mass of the sodium salt of one added sulfate group). The resulting product, known as PSGal-64, is more highly sulfated than compound 4e, described above and in Table 1.

### Example 7

### Matrix-Assisted Laser Desorption Ionization Time-of-Flight (MALDI-TOF) Mass Spectrometry

MALDI-TOF MS is done on a Perseptive Biosystems Voyager DE-PRO spectrometer. Spectra (100) of negatively charged sugars are generated in linear, negative ion mode using THAP matrix; or in linear, positive ion mode using IAA. Glycodendrimer spectra (200) are generated in linear, positive ion mode using an IAA matrix (20 mg/mL in DMF, diluted 11:1 with a 1 mM aqueous solution of glycodendrimer to yield a matrix to analyte ratio of ˜1000:1) as described in Woller, E. K. and Cloninger, M. J., 2001, Biomacromolecules 2, 1052-1054. In some instances, negative ion mode is used to detect the sulfated glycodendrimers. Average molecular weights and polydispersities of the glycodendrimers are calculated using Data Explorer version 4.0 (Applied Biosystems). Macros within this software allow for the calculation of Mn (the number-average molecular weight), Mw (the weight-average molecular weight), and the polydispersity (PD) from the ratio of Mw to Mn according to the following equations:

Mn=Σ(NiMi)/ΣNi

Mw=Σ(NiMi 2)/ΣNiMi

where Ni and Mi represent signal intensity and mass at point i, respectively. For detailed methods, see Zhu, H., et al., 1998, American Society for Mass Spectrometry 9, 275-281 and Zhu, T., et al., 1993, Science 261, 1179-1181.

### Example 8

Nuclear Magnetic Resonance (NMR) Analyses. All spectra are obtained on a Bruker DRX-400 NMR spectrometer operating in the quadrature mode at 25.0° C. using a triple-axis-gradient broadband inverse probe. 1H chemical shifts are referenced indirectly to TMS (0 ppm) via DSS (sodium-2,2-dimethyl-2-silapentane-5-sulfonate), a secondary reference. 13C chemical shifts are referenced indirectly to TMS (0 ppm) via acetone (30.89 ppm and 215.9 ppm), a secondary reference. All samples are dissolved in D2O. One bond 1H-13C correlation 2D spectra are obtained using the gradient selected version of the phase-sensitive HMQC experiment. Long range (2 and 3 bond) 1H-13C correlation 2D spectra are obtained using the gradient-selected version of the magnitude-mode HMBC experiment. When necessary for assignment purposes, gradient-selected Double-Quantum-Filtered COSY experiments are used to verify 1H-1H couplings.

### Example 9

Kinetic analysis of the interaction between rgpl20 IIIB and the synthesized glycodendrimers.

SPR is used to study the binding kinetics of rgpl20 to the different derivatized dendrimers. Sensorgrams are analyzed by fitting the data to a 1:1 (Langmuir) binding model and kinetic constants determined using the BiaEvaluation 3.2 software. Rate constants and affinity constants for the interaction of rgpl20 with each of the glycodendrimers and DxS are shown in Table 2. Overall, the affinities of rgpl20 for the various glycodendrimers are in the nanomolar range, with the SGalderivatized denrimers (4c-4e) displaying somewhat stronger affinities than their Gal-derivatized dendrimer counterparts (2c-2e).

TABLE 2

| Compound | Kₛ | K_{d} | K_{D} (M) |
|---|---|---|---|
| 1c GC-16mer | 1.73 × 10⁵ | 1.03 × 10⁻³ | 5.94 × 10⁻⁹ |
| 1d GC-32mer | 3.70 × 10⁵ | 5.61 × 10⁻⁴ | 1.52 × 10⁻⁹ |
| 1e GC-64mer | 1.42 × 10⁵ | 9.60 × 10⁻⁴ | 6.78 × 10⁻⁹ |
| 2c Gal-16mer | 4.46 × 10⁴ | 1.35 × 10⁻³ | 3.02 × 10⁻⁸ |
| 2d Gal-32mer | 1.89 × 10⁵ | 1.43 × 10⁻³ | 7.57 × 10⁻⁹ |
| 2e Gal-64mer | 1.86 × 10⁵ | 1.15 × 10⁻³ | 6.18 × 10⁻⁹ |
| 4c SGal-32mer | 9.56 × 10⁴ | 3.09 × 10⁻⁴ | 3.23 × 10⁻⁹ |
| 4d SGal-32mer | 6.27 × 10⁵ | 1.18 × 10⁻⁴ | 1.89 × 10⁻¹⁰ |
| 4e SGal-32mer | 5.11 × 10⁵ | 9.04 × 10⁻⁴ | 1.77 × 10⁻⁹ |
| DxS (50 kDa) | 2.83 × 10⁶ | 6.49 × 10⁻⁵ | 2.29 × 10⁻¹¹ |

SPR experiments are done at 25° C. using a Biacore 3000 system and CM5 sensor chips (Biacore, Uppsala, Sweden). Glycodendrimers, 1c-1e and 2c-2e, are amine coupled to the sensor chip according to the manufacturer's protocol, using the free amines that are not functionalized on the glycodendrimers. In order to obtain a low density surface suitable for affinity analysis, 350-500 RUs of each glycodendrimer are immobilized. Varying concentrations of recombinant gpl20 (rgp 120), 62.5 nM-0.98 nM, are injected simultaneously over a blank control surface and the immobilized glycodendrimers at a flow rate of 30 μl/min for 3 minutes in HBS-EP buffer (10 mM HEPES, 150 mM NaCl, 3 mM EDTA, 0.005% P-20 surfactant, pH 7.4). Following the injection, the dissociation of the rgpl20-glycodendrimer complex is measured for 10 min. Any remaining bound rgpl20 is removed by a 1 min injection of 4M KCl. This regeneration step prepared the surface for the next injection of rgpl20. Each concentration of rgpl20 is injected in duplicate over the immobilized glycodendrimer, with a representative sensorgram chosen for analysis.

The sulfated glycodendrimers, 4c-4e, did not couple to the CM5 chip, presumably due to repulsion of the negatively charged sulfate groups by the negatively charged carboxyl groups on the carboxymethylated dextran matrix. Therefore, approximately 3000 RUs of rgpl20 is amine coupled to the CM5 sensor chip and the potential ligand used as analyte. DxS (50 kDa, 12.5 nM-0.39 nM), 4c, 4d, or 4e (89 nM-0.36 nM) are injected simultaneously over a blank control surface and the immobilized rgpl20 at a flow rate of 30 μl/min, for 3 min. Dissociation of the complex is measured for 10 min, followed by a 1 min injection of 4M KCl to regenerate the rgpl20 surface. Non-specific binding is accounted for by subtraction of the blank control surface from each of the test surfaces. Subtracted sensorgrams are then analyzed by fitting the data to a 1:1 (Langmuir) binding model and kinetic constants determined using the BiaEvaluation 3.2 software.

### Example 10

### Cell Culture

U373-MAGI-CXCR4 and U373-MAGI-CCR5 cells (catalog #3596 and #3597, respectively) are indicator cells as described in Vodicka, M. A., et al., 1997, Virology. 233:193-8, obtainable from the AIDS Research and Reference Reagent Program, Division of AIDS, NIAID, NIH. Cells are propagated in ‘Selection Media’ consisting of DMEM supplemented with 10% FCS, 0.4 mg/ml L-glutamine, penicillin and streptomycin (0.08 mg/ml each), 0.05% sodium bicarbonate, 0.2 mg/ml G418, 0.1 mg/ml hygromycin B, and 1.0 ug/ml puromycin. ‘Culture Media’ for virus inhibition assays consists of DMEM with 10% FCS and 0.05% sodium bicarbonate. Cells are grown at 37° C. in an atmosphere of 95% air/5% CO2.

### Example 11

### Viral Inhibition Assays

Cells, in Selection Media, are plated in 96-well tissue culture plates at a density of 1.0×10⁴ cells per well. One day later, commercially available (Advanced Biotechnologies, Columbia, Md.) concentrated cell-free viral preparations of HIV-1 Ba-L (R5), HIV-1 IIIB (X4), NL4-3 (X4), and 89.6 (X4/R5) are diluted 1:400, 1:200, 1:100 and 1:10, respectively, in ‘Culture Media’. Glycodendrimer and DxS 2 mg/ml stock solutions are made up in culture media, and serially diluted with culture media in separate sterile V-bottom 96-well microtiter plates, to a final volume of 30 microliters per well. This is followed by the addition of 30 microliters of the diluted virus, and incubation at 37° C. for ˜10-20 minutes. Selection Media is then removed from the plated cells, and 50 microliters of virus or virus plus potential inhibitor is added to the plated cells. The virus- or virus plus inhibitor-exposed cells are then incubated at 37° C. in an atmosphere of 95% air/5% CO2 for 2 hr. After the absorption period, 200 microliters of Culture Media is added to the wells and the cells allowed to grow for an additional 40-48 hrs at 37° C. in an atmosphere of 95% air/5% CO2 for 2 hr. Cells treated with culture media serve as negative controls, and cells treated with virus only are positive controls. All measurements are done in quadruplicate.

After the 48 hr incubation, culture media is removed from the wells, the cells washed with 200 μl of PBS, and β-galactosidase activity measured using the Galacto-Star™ β-Galactosidase Reporter Gene Assay System from Applied Biosystems (Foster City, Calif.), according to the manufacturer's instructions. Briefly, after the cells are washed with PBS, 10 microliters of lysis solution is added to each well and the plate incubated for 10 min at 37° C. The Galacto-Star™ substrate is diluted 1:50 and 100 microliters added to each well containing cell lysate. Well contents are mixed and 90 microliters were removed and added to a 96-well opaque luminometer plate. One hour after the Galacto-Star™ substrate is added, luminescence is determined using a luminometer. Percent inhibition is determined as [(L_{no inhibitor}-L_{inhibitor})/L_{no inhibitor}]×100. The effective concentration that inhibits 50% of viral infectivity (EC50) is determined by plotting the percent inhibition versus the log of the concentration of the potential inhibitor.

### Example 12

Inhibition of HIV-1 Infection

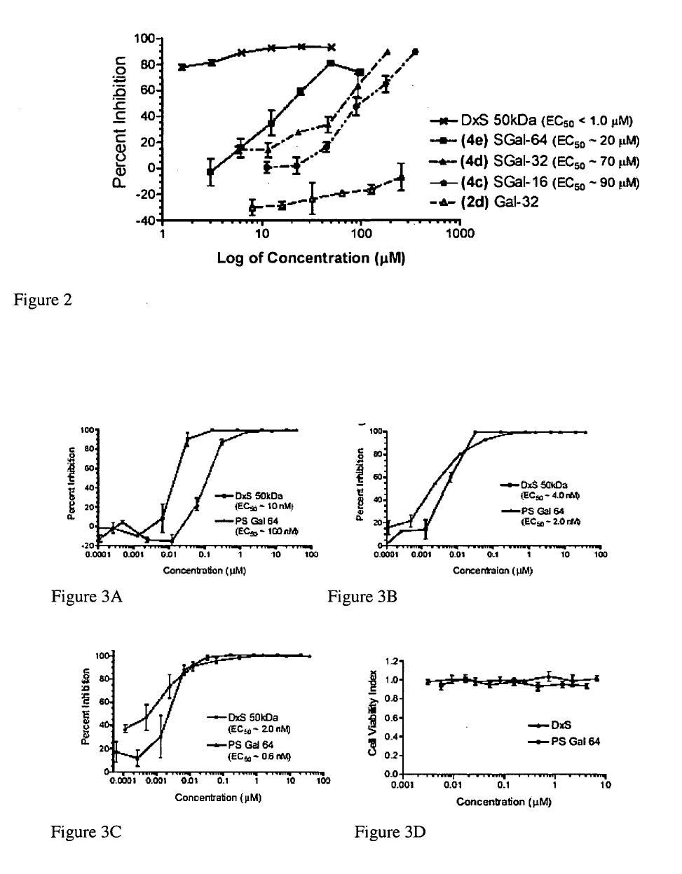

Glycodendrimers 1c-1e, 2c-2e, and 4c-4e, and DxS 50 kDa, are tested for their ability to inhibit HIV-1 BaL (R5-tropic) infection of U373-MAGI-CCR5 cells, and their affect on cell viability. The observed EC50s for the sulfated glycodendrimers 4c, 4d, and 4e are 90 micromolar, 70 micromolar, and 20 micromolar, respectively, FIG. 2. An increase in inhibition is seen with increasing size of the sulfated glycodendrimers. DxS is a potent inhibitor of infection by HIV-1 BaL with an EC50 of 1.6 micromolar. The galactose functionalized glycodendrimers (1c-1e and 2c-2e) have less inhibitory effect. The 50% inhibition level is not reached at concentrations of galactose-derivatized dendrimer as high as 2.5 milligrams/milliliter.

### Example 13

Inhibition of HIV-1 Infection

Multivalent glycodendrimers bearing sulfated galactose residues are synthesized and tested for their ability to inhibit HIV-1 infection of cultured reporter cells as described above. Viral inhibition assays using three HIV-1 isolates, HIV-1 IIIB (X4), NL4-3 (X4), and 89.6 (X4/R5], indicate PS Gal 64mer glycodendrimer is an effective, inhibitor of HIV-1 infectivity, with nanomolar EC50s as shown in FIG. 3. FIGS. 3A-D generally shows inhibition of HIV-1 infectivity and assessment of cytotoxicity. In particular, FIG. 3A shows inhibition of HIV-1 IIIB infection of U373-MAGI-X4 cells by DxS and PS Gal 64mer. FIG. 3B shows inhibition of HIV-1 NL4-3 infection of U373-MAGI-X4 cells by DxS and PS Gal 64mer. FIG. 3C shows inhibition of HIV-1 89.6 infection of U373-MAGI-R5 cells by DxS and PS Gal 64. FIG. 3D shows cytotoxicity of DxS and PS Gal 64 on U373-MAGI-X4 cells. Measurements are done in quadruplicate. Error bars represent the SD with n=3 or n=4. Replicate experiments with HIV-1 IIIB give similar results.

### Example 14

Inhibition of HIV-1 Infection

Dendrimer conjugates are made which are functionalized as described using glucosylthiopropionic acid, galactoseβ1-4glucose-thiopropionic acid or galactoseα1-4 galactoseβ1-4glucose-thiopropionic acid each including sulfur and/or phosphate containing groups as detailed herein. Each is tested in an assay of inhibition of viral infectivity as in Example 13. Similar results are obtained.

### Example 15

Inhibition of HIV-1 Infection

### Example 16

Dendrimer conjugates are made which are functionalized as described using sialic acid(α2-3)galactose(β1-4)glucosyl thiopropionic acid including sulfur and/or phosphate containing groups as detailed herein. The compositions are tested in an assay of inhibition of viral infectivity as in Example 13. Similar results are obtained.

Cytotoxicity

The effect of the glycodendrimers and control compounds on cell viability is determined using the CellTiter 96® AQueous Cell Proliferation Assay (Promega, Madison, Wiss.). In this viability assay, mitochondrial dehydrogenases in metabolically active cells catalyze the conversion of MTS into a formazan product. The absorbance of formazan is read at 490 nm and is directly proportional to the number of viable cells. Assays are performed according to the manufacturer's instructions.

Replicate 96-well plates of each of the HIV infection inhibition assay plates are set up and treated identically, with the exception that no virus is added to the cells. After the 48 hr incubation, the cell culture media is removed, cells washed with PBS, and 100 μl of culture media is added to each well. The MTS reagent is mixed with the electron coupling reagent phenazine methosulfate, PMS, according to the instructions, and 25-100 μl of the mixture is added to the cells. The 96-well replicate plates are incubated for 3 hr at 37° C., in an atmosphere of 95% air/5% CO2, and absorbance read on an ELISA plate reader at 490 nm. Cell viability indices are calculated by dividing the average absorbance of non-treated cells (negative control wells) into the average absorbance obtained for each concentration of each compound tested. Therefore, non-treated cells would have a viability index of 1.0 while that for cells exposed to cytotoxic compounds would be less than 1.0. The index obtained for each concentration of a compound is then used to correct for inhibition due to cell death induced by a potential inhibitor. None of the compounds tested including 2d, 4c-4e, 2d, PS Gal-64 and DxS are cytotoxic to the cultured indicator cells at the highest concentrations tested (2.5 milligrams/milliliter).

Any patents or publications mentioned in this specification are indicative of the levels of those skilled in the art to which the invention pertains. These patents and publications are herein incorporated by reference to the same extent as if each individual publication was specifically and individually indicated to be incorporated by reference. In particular, this application claims priority of U.S. Provisional Patent Applications 60/454,210 filed Mar. 10, 2003, and 60/545,072, filed Feb. 17, 2004, both of which are incorporated herein by reference.

One skilled in the art will readily appreciate that the present invention is well adapted to carry out the objects and obtain the ends and advantages mentioned, as well as those inherent therein. The apparatus and methods described herein are presently representative of preferred embodiments, exemplary, and not intended as limitations on the scope of the invention. Changes therein and other uses will occur to those skilled in the art. Such changes and other uses are encompassed within the spirit of the invention as defined by the scope of the claims.

We claim:

1. A composition comprising:
a compound having the formula:
[(X)—(Y)]ₚ-Z
where X is 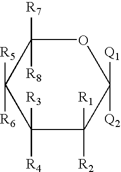
where Q1 and Q2 are each independently H, a bond to Y, or a bond to Z, where at least one of Q1 or Q2 is a bond to Y or a bond to Z; where R1, R4, R6 and R8 are each H; R2, R3, and R5 are each independently OH, OSO3D, or OPO3D, and R7 is CH2OH, CH2OSO3D or CH2OPO3D; where D is H or a cation selected from the group consisting of: alkali metal cations, alkaline earth metal cations, ammonium cations, quaternary ammonium cations and amine cations; and where at least one of R2, R3, R5 and R7 is a sulfur or phosphate containing group;
where Y is an optional linker, Z is a multivalent support, and p is an integer in the range of 1-2000 inclusive.

2. The composition of claim 1 where p is 2-2000 inclusive, and where at least one of R2, R3, R5 and R7 on each X independently is a sulfur or phosphate containing group.

3. The composition of claim 1 where p is 3-525 inclusive, and where at least one of R2, R3, R5 and R7 on each X independently is a sulfur or phosphate containing group.

4. The composition of claim 1 having an average of 1-4 sulfur or phosphate containing groups per moiety X.

5. The composition of claim 1 having an average of 2-3 sulfur or phosphate containing groups per moiety X.

6. The composition of claim 1 wherein Z is a dendrimer.

7. The composition of claim 1 wherein Z is selected from the group consisting of: a protein, a lipid, a carbohydrate, a synthetic polymer, a natural polymer and combinations thereof.

8. A composition comprising:
a compound having the formula:
[(X)—(Y)]ₚ-Z
where X is 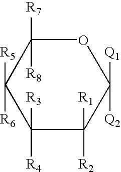
where Q1 and Q2 are each independently H, a bond to Y, or a bond to Z, where at least one of Q1 or Q2 is a bond to Y or a bond to Z; where R1, R4, R5 and R8 are each H; R2, R3, and R6 are each independently OH, OSO3D, or OPO3D, and R7 is CH2OH, CH2OSO3D, CH2OPO3D; where D is H or a cation selected from the group consisting of: alkali metal cations, alkaline earth metal cations, ammonium cations, quaternary ammonium cations and amine cations; and where at least one of R2, R3, R6 and R7 is a sulfur or phosphate containing group; where Y is an optional linker, Z is a multivalent support, and p is an integer in the range of 1-2000.

9. The composition of claim 8 where p is 2-2000 inclusive and where at least one of R2, R3, R6 and R7 on each X independently is a sulfur or phosphate containing group.

10. The composition of claim 8 where p is 3-525 inclusive and where at least one of R2, R3, R6 and R7 on each X independently is a sulfur or phosphate containing group.

11. The composition of claim 8 having an average of 1-4 sulfur or phosphate containing groups per moiety X.

12. The composition of claim 8 having an average of 2-3 sulfur or phosphate containing groups per moiety X.

13. The composition of claim 8 wherein Z is a dendrimer.

14. The composition of claim 8 wherein Z is selected from the group consisting of: a protein, a lipid, a carbohydrate, a synthetic polymer, a natural polymer and combinations thereof.

15. A composition comprising:
a compound having the formula:
[(X)—(Y)]ₚ-Z
where X is 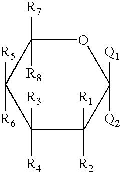
where Q1 and Q2 are each independently H, a bond to Y, or a bond to Z, where at least one of Q1 or Q2 is a bond to Y or a bond to Z; where R1, R2, R3, R4, R5 and R6 are each independently H, OH, COOH, sialic acid, OSO3D, or OPO3D; R7 and R8 are each independently H, OH, COOH, sialic acid, CH2OH, CH2OSO3D, CH2OPO3D, OSO3D, or OPO3D, where D is H or a cation selected from the group consisting of: alkali metal cations, alkaline earth metal cations, ammonium cations, quaternary ammonium cations and amine cations; and where at least one of R1, R2, R3, R4, R5, R6, R7 and R8 is a sulfur or phosphate containing group; where Y is an optional linker, Z is a multivalent support, and p is an integer in the range of 1-2000.

16. The composition of claim 15 where p is 2-2000 inclusive and where at least one of R1, R2, R3, R4, R5, R6, R7 and R8 on each X independently is a sulfur or phosphate containing group.

17. The composition of claim 15 where p is 3-525 inclusive and where at least one of R1, R2, R3, R4, R5, R6, R7 and R8 on each X independently is a sulfur or phosphate containing group.

18. The composition of claim 15 having an average of 1-4 sulfur or phosphate containing groups per moiety X.

19. The composition of claim 15 having an average of 2-3 sulfur or phosphate containing groups per moiety X.

20. The composition of claim 15 wherein Z is a dendrimer.

21. The composition of claim 15 wherein Z is selected from the group consisting of: a protein, a lipid, a carbohydrate, a synthetic polymer, a natural polymer and combinations thereof.

22. A composition comprising:
a compound having the formula:
[(X)—(Y)]ₚ-Z
where X is 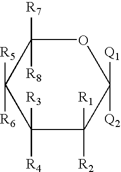
where Q1 and Q2 are each independently H, a bond to Y, or a bond to Z, where at least one of Q1 or Q2 is a bond to Y or a bond to Z; where the bond to Y is a bond to an atom selected from the group consisting of: S, P, N, or C; where R1, R2, R3, and R4 are each independently H, OH, COOH, sialic acid, a saccharyl group, OSO3D, or OPO3D; where R7 and R8 are each independently H, CH2OH, COOH, sialic acid, a saccharyl group, CH2OSO3D, CH2OPO3D, OSO3D, or OPO3D; where R5 and R6 are each independently H or a saccharyl group having the formula: 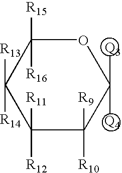
where one of the variables Q3 and Q4 is O and the other is H; where R9, R10, R11, R12, R13 and R14 are each independently H, OH, COOH, sialic acid, a saccharyl group, OSO3D, or OPO3D; where R15 and R16 are each independently H, OH, COOH, sialic acid, a saccharyl group, CH2OH, CH2OSO3D, CH2OPO3D, OSO3D, or OPO3D; where D is H or a cation selected from the group consisting of: alkali metal cations, alkaline earth metal cations, ammonium cations, quaternary ammonium cations and amine cations; where at least one of R1, R2, R3, R4, R5, R6, R7, R8 R9, R10, R11, R12, R13, R14, R15 and R16 is a sulfur or phosphate containing group; and where Y is an optional linker, Z is a multivalent support, and p is an integer in the range of 1-2000.

23. The composition of claim 22 where p is 2-2000 inclusive and where at least one of R1, R2, R3, R4, R5, R6, R7 and R8 on each X independently is a sulfur or phosphate containing group.

24. The composition of claim 22 where p is 3-525 inclusive and where at least one of R1, R2, R3, R4, R5, R6, R7 and R8 on each X independently is a sulfur or phosphate containing group.

25. The composition of claim 22 having an average of 1-4 sulfur or phosphate containing groups per moiety X.

26. The composition of claim 22 having an average of 2-3 sulfur or phosphate containing groups per moiety X.

27. The composition of claim 22 wherein Z is a dendrimer.

28. The composition of claim 22 wherein Z is selected from the group consisting of: a protein, a lipid, a carbohydrate, a synthetic polymer, a natural polymer and combinations thereof.

29. A composition comprising:
a compound having the formula:
[(X)—(Y)]ₚ-Z
where X is 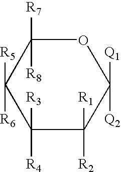
where Q1 and Q2 are each independently H or a bond to Y, where at least one of Q1 or Q2 is a bond to Y; and where R1, R2, R3, and R4 are each independently H, OH, COOH, sialic acid, a saccharyl group, OSO3D, or OPO3D; R7 and R8 are each independently H, COOH, sialic acid, a saccharyl group, CH2OH, CH2OSO3D, CH2OPO3D, OSO3D, or OPO3D; and R5 and R6 are each independently H or a saccharyl group having the formula: 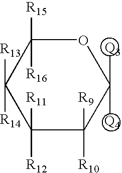
where one of the variables Q3 and Q4 is oxygen and the other is H; and where R9, R10, R11, R12, R13 and R14 are each independently H, OH, COOH, sialic acid, a saccharyl group, OSO3D, or OPO3D; and R15 and R16 are each independently H, OH, COOH, sialic acid, a saccharyl group, CH2OH, CH2OSO3D, CH2OPO3D, OSO3D, or OPO3D; where D is H or a cation selected from the group consisting of: alkali metal cations, alkaline earth metal cations, ammonium cations, quaternary ammonium cations and amine cations; and where at least one of R9, R10, R11, R12, R13, R14, R15 and R16 is a sulfur or phosphate containing group; and where Y is an optional linker, Z is a multivalent support, and p is an integer in the range of 1-2000.

30. The composition of claim 29 where p is 2-2000 inclusive and where at least one of R1, R2, R3, R4, R5, R6, R7 and R8 on each X independently is a sulfur or phosphate containing group.

31. The composition-of claim 29 where p is 3-525 inclusive and where at least one of R1, R2, R3, R4, R5, R6, R7 and R8 on each X independently is a sulfur or phosphate containing group.

32. The composition of claim 29 having an average of 1-4 sulfur or phosphate containing groups per moiety X.

33. The composition of claim 29 having an average of 2-3 sulfur or phosphate containing groups per moiety X.

34. The composition of claim 29 wherein Z is a dendrimer.

35. The composition of claim 29 wherein Z is selected from the group consisting of: a protein, a lipid, a carbohydrate, a synthetic polymer, a natural polymer and combinations thereof.

36. A composition comprising:
a compound having the formula:
[(X)—(Y)]ₚ-Z
where X is 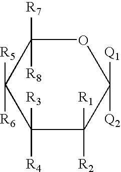
where Q1 and Q2 are each independently H or a bond to Y, where at least one of Q1 or Q2 is a bond to Y; and where R1, R2, R3, and R4 are each independently H, OH, COOH, sialic acid, a saccharyl group, OSO3D, or OPO3D; R7 and R8 are each independently H, OH, COOH, sialic acid, a saccharyl group, CH2OH, CH2OSO3D, CH2OPO3D, OSO3D, or OPO3D; and R5 and R6 are each independently H or a saccharyl group having the formula: 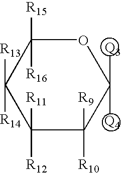
where one of the variables Q3 and Q4 is oxygen and the other is H; where R9, R10, R11, and R12 are each independently H, OH, COOH, sialic acid, a saccharyl group, OSO3D, or OPO3D; where R15 and R16 are each independently H, OH, COOH, sialic acid, a saccharyl group, CH2OH, CH2OSO3D, CH2OPO3D, OSO3D, or OPO3D; where R13 and R14 are each independently H or a saccharyl group having the formula: 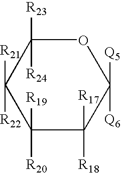
where one of the variables Q5 and Q6 is oxygen and the other is H; and where R17, R18, R19, R20, R21 and R22 are each independently a saccharyl group, H, OH, COOH, sialic acid, OSO3D, or OPO3D; and R23 and R24 are each independently a saccharyl group, H, OH, COOH, sialic acid, CH2OH, CH2OSO3D, CH2OPO3D, OSO3D, or OPO3D; where D is H or a cation selected from the group consisting of: alkali metal cations, alkaline earth metal cations, ammonium cations, quaternary ammonium cations and amine cations; and where at least one of R1, R2, R3, R4, R5, R6, R7, R8 R9, R10, R11, R12, R13, R14, R15, R16 R17, R18, R19, R20, R21, R22, R23 and R24 is a sulfur or phosphate containing group; and where Y is an optional linker, Z is a multivalent support, and p is an integer in the range of 1-2000.

37. The composition of claim 36 where p is 2-2000 inclusive and where at least one of R1, R2, R3, R4, R5, R6, R7 and R8 on each X independently is a sulfur or phosphate containing group.

38. The composition of claim 36 where p is 3-525 inclusive and where at least one of R1, R2, R3, R4, R5, R6, R7 and R8 on each X independently is a sulfur or phosphate containing group.

39. The composition of claim 36 having an average of 1-4 sulfur or phosphate containing groups per moiety X.

40. The composition of claim 36 having an average of 2-3 sulfur or phosphate containing groups per moiety X.

41. The composition of claim 36 wherein Z is a dendrimer.

42. The composition of claim 36 wherein Z is selected from the group consisting of: a protein, a lipid, a carbohydrate, a synthetic polymer, a natural polymer and combinations thereof.

43. A pharmaceutical composition comprising a compound according to claim 1; and
a pharmaceutical carrier or excipient.

44. A pharmaceutical composition comprising a compound according to claim 8; and
a pharmaceutical carrier or excipient.

45. A pharmaceutical composition comprising a compound according to claim 15; and
a pharmaceutical carrier or excipient.

46. A pharmaceutical composition comprising a compound according to claim 22; and
a pharmaceutical carrier or excipient.

47. A pharmaceutical composition comprising a compound according to claim 29; and
a pharmaceutical carrier or excipient.

48. A pharmaceutical composition comprising a compound according to claim 36; and
a pharmaceutical carrier or excipient.

49. A process for inhibiting interaction of a ligand and a receptor, the process comprising the steps of:
providing a composition as claimed in claim 1; and
introducing the composition into an environment containing the ligand or the receptor, such that interaction of the ligand and the receptor is inhibited by the composition.

50. The process of claim 49 wherein the ligand is a virus or a component of a virus.

51. The process of claim 49 wherein the ligand is a cell or a component of a cell.

52. The process of claim 49 wherein the receptor is a cell or a component of a cell.

53. The process of claim 50 wherein the virus is a virus of the family Retroviridae.

54. The process of claim 50 wherein the virus is an HIV.

55. The process of claim 49 wherein the environment is a human or non-human animal body.

56. The process of claim 49 wherein the ligand is a sperm cell and the receptor is an ovum.

57. The process of claim 49 wherein the receptor is endogenous to the body of an individual subject and the ligand is exogenous.

58. A process for inhibiting interaction of a ligand and a receptor, the process comprising the steps of:
providing a composition as claimed in claim 8; and
introducing the composition into an environment containing the ligand or the receptor, such that interaction of the ligand and the receptor is inhibited by the composition.

59. A process for inhibiting interaction of a ligand and a receptor, the process comprising the steps of:
providing a composition as claimed in claim 15; and
introducing the composition into an environment containing the ligand or the receptor, such that interaction of the ligand and the receptor is inhibited by the composition.

60. A process for inhibiting interaction of a ligand and a receptor, the process comprising the steps of:
providing a composition as claimed in claim 22; and
introducing the composition into an environment containing the ligand or the receptor, such that interaction of the ligand and the receptor is inhibited by the composition.

61. A process for inhibiting interaction of a ligand and a receptor, the process comprising the steps of:
providing a composition as claimed in claim 29; and
introducing the composition into an environment containing the ligand or the receptor, such that interaction of the ligand and the receptor is inhibited by the composition.

62. A process for inhibiting interaction of a ligand and a receptor, the process comprising the steps of:
providing a composition as claimed in claim 36; and
introducing the composition into an environment containing the ligand or the receptor, such that interaction of the ligand and the receptor is inhibited by the composition.

63. A process of therapeutic or prophylactic anti-viral treatment, comprising:
administering an effective amount of a compound as claimed in claim 1 to an individual human or non-human animal in need of such therapeutic or prophylactic anti-viral treatment.

64. The process of claim 63 wherein the individual human or non-human animal is at risk of retroviral infection.

65. The process of claim 63 wherein the individual human or non-human animal is infected with a retrovirus.

66. A commercial package comprising:
a composition as claimed in claim 1; and
instructions for use.
